(19) United States
(12) Patent Application Publication
(10) Pub. No.: US 2003/0228599 A1
(43) Pub. Date: Dec. 11, 2003

(54) Genomic profiling: a rapid method for testing a complex biological sample for the presence of many types of organisms

(76) Inventor: Don Straus, Cambridge, MA (US)

Correspondence Address: CLARK & ELBING LLP, 101 FEDERAL STREET, BOSTON, MA, 02110, US

(21) Appl. No.: 10/395,039

(22) Filed: Mar. 21, 2003

Related U.S. Application Data
(63) Continuation of application No. 09/333,110, filed on Jun. 15, 1999, now abandoned.

(51) Int. Cl. C12Q 1/68; G06F 19/00; G01N 33/48; G01N 33/50; C12P 19/34

(52) U.S. Cl. 435/6; 702/20; 435/91.2

(57) ABSTRACT

The invention provides a method, referred to as genomic profiling, which simultaneously scans a complex biological sample for the presence of nucleic acid sequences (including genomic difference sequences, group-specific sequences, and DNA polymorphisms) that are diagnostic of numerous different types of organisms. Also included in the invention are probes, detection ensembles, and related molecules for use in the methods of the invention.

### BACKGROUND OF THE INVENTION

The invention relates to obtaining genetic information from complex biological samples, such as bodily samples (e.g., blood, urine, sputum, and feces). It is medically important to identify infectious organisms in such samples for optimum treatment of infections and for maintaining public health. Determining whether a patient suffers from a hereditary disease and forensic identification also relies heavily on analysis of genetic information in bodily samples.

Although current procedures for diagnosing infectious agents include a complex battery of hundreds of tests, a large fraction of infectious organisms routinely escape detection. For example, the success rate is only about half in attempts to determine the infectious agent in patients with pneumonia, the most common cause of death by infectious disease in the United States.

Many diseases, such as pneumonia, meningitis, and acute gastrointestinal illness, are characterized by a set of symptoms (a “presentation”) that can be caused by a multitude of infectious agents. There is no single test that scans for all of the pathogens that commonly cause such diseases. (I refer to such a test as a “presentation-specific test.”) Current procedures often test for the presence of only a single type of pathogenic organism. This is problematic, as many different tests must often be carried out on a sample, increasing the cost, required time for identification, and likelihood of error.

Also, many procedures are too expensive for routine use. For example, it may cost hundreds of dollars to test for a particular virus. This cost must be weighed by health care providers, especially in light of the fact that multiple tests are likely to be required for identification of the infectious agent.

Most current diagnostic tests require that the infectious agent be cultured to attain a large number of organisms. Unfortunately, many types of organisms cannot be routinely cultured in hospital laboratories. Most viruses and parasites and many bacteria fall into this category. For organisms that can be cultured, critical time is lost by culturing, which can take days or even weeks. Thus, the life of a patient with, for example, bacterial meningitis may critically depend on immediate treatment, but optimal treatment may require time consuming and life threatening delays, due to culturing. Other infectious agents, such as the bacterium that causes tuberculosis, generally require weeks to grow in culture. The delay in identification (and optimum treatment) can lead to a patient with tuberculosis infecting many others with the highly contagious disease.

Current diagnostic tests practiced in hospitals yield only crude identification of the class of organism present in a sample. In many cases, it is difficult to distinguish a pathogenic organism from a closely related non-pathogen.

Furthermore, to identify a pathogen, a sample may have to undergo many tests, in several different laboratories, carried out by several personnel, each with a different type of specialized training. The expense required for the necessary specialists is a major drain on the budget of diagnostics laboratories. Also, splitting samples among various laboratories introduces another source of error, and transport may be problematic if pathogen viability is required for the test.

Thus, there is a need for a new type of test that is presentation-specific (i.e., comprehensive), efficiently checks for the presence of a large number of organisms from various diverse groups, can be performed in a relatively short time (such as a few hours), uses a single test format, and leads to high-resolution identification of pathogens.

Obtaining precise genetic information from biological samples can be informative about the identity and medically relevant attributes of the organisms present in the samples. This is because every type of organism has a unique genomic DNA sequence, due to evolutionary divergence.

The causes of change in DNA sequence over time include battery by cosmic rays, modification by chemical mutagens, mistakes in normal DNA replication, rearrangement by genetic recombination, and invasion by viruses, plasmids, and transposable genetic elements. As a result, single base changes accumulate, segments of sequences are deleted, segments of sequences are inserted, and chromosomes rearrange. Thus, genomes are mosaics of conserved sequences (i.e., sequences that are common to diverse taxa) and divergent sequences that are the result of the types of changes enumerated above. Methods that test for unique genomic signatures, or fingerprints, are therefore useful for identifying organisms.

Numerous methods have been developed for obtaining DNA fingerprints of infectious organisms. These include restriction fragment length polymorphism (RFLP) analysis, amplified fragment length polymorphism (AFLP) analysis, pulsed-field gel electrophoresis, arbitrarily-primed polymerase chain reaction (AP-PCR), repetitive sequence-based PCR, ribotyping, and comparative nucleic acid sequencing. These methods are generally too slow, expensive, irreproducible, and technically demanding to be used in most diagnostic settings. All of the above-mentioned methods generally require that a cumbersome gel electrophoretic step be used, that the pathogen be grown in culture, that its genomic DNA be purified, and that the sample not contain more than one type of organism (this rules out direct testing of complex medical samples). The same limitations (with the exception of the requirement for gel electrophoresis) apply to recently developed methods for high resolution strain identification relying on sample hybridization to high density microarrays (Salazar et al., Nucleic Acids Res. 24:5056-5057, 1996; Troesch et al., J. Clin. Microbiol. 37:49-55, 1999; Lashkari et al., Proc. Natl. Acad. Sci. U.S.A. 94:13057-13062, 1997). Furthermore, these new hybridization methods can be technically demanding because they generally require discrimination between hybridization to small oligonucleotides with various degrees of mismatching. A method based on the presence or absence of larger DNA sequences would provide a more robust, and therefore more clinically useful, diagnostic assay. Precise genetically-based identification, in the form of DNA fingerprints, is critical for tracking and controlling infectious outbreaks in communities and in hospitals. Therapeutically, fingerprinting, especially if it could be offered in a rapid, culture-independent test, could save lives by determining which antibiotic to administer more rapidly than can be determined by current practices.

Methods have also been developed for testing a sample for the presence of several types of diverse organisms at once. Note that such methods are, as yet, generally not suited for fingerprinting—that is, for distinguishing between closely related organisms within a species. One method for testing for the presence of several organisms at once, without requiring culturing, is multiplex PCR. A major problem of multiplex PCR, along with other multiplexed amplification methods, is that it is difficult to amplify many sequences simultaneously (amplification artifacts begin to accumulate as more primer sequences are included). Because of the limitation on numbers of sequences that can be tested for using multiplex PCR, it is very difficult to arrive at a robust multiplexed test for numerous different sequences that occur in numerous different types of organisms. Thus, one of the best examples of applying multiplex PCR to test simultaneously for phylogenetically disparate organisms checks for only nine sequences, which is not nearly enough to provide for a presentation-specific test (Grondahl et al., J. Clin. Microbiol. 37:1-7, 1999). Furthermore, due to the limitation in number of diagnostic probes that can be used (only one sequence per type of organism was tested) this test lacks redundancy (important for reproducibility) and offers only crude identification of the infectious agents. Multiplex PCR is also sensitive to inhibitors present in most medical samples, and requires technically demanding sample preparation for reliable results.

One method to genetically identify an organism involves testing for the presence of a sequence (or set of sequences) that is unique to the particular type of organism. Such sequences are called identification (ID) sequences. To determine the presence of human immunodeficiency virus, for example, one tests for the presence of a DNA sequence that is uniquely present in members of this group of viruses. As another example, one strain of Escherichia coli might be harmless when present in the human gastrointestinal tract, while the presence of another strain of E. coli might be life threatening. Although such strains may be very closely related, they can be distinguished by detecting variation in their DNA sequences.

To distinguish an organism from closely related relatives, it is useful to test for the presence of members of a set of DNA sequences that occur in unique combinations in each strain from within a group. Such sequences, termed genomic difference sequences, have been described in the literature, e.g., in Straus (“Genomic Subtraction,” In PCR Strategies, Innes et al., Eds., p. 220-236 (Academic Press Inc., San Diego, 1995)), which is hereby incorporated by reference. Genomic difference sequences are DNA sequences that hybridize to the genome of one organism, but not to the genome of a different, but closely related, organism. As is described in Straus (1995, supra), genomic difference sequences can be prepared, for example, by carrying out subtractive hybridization with the genomes of two distinct organisms. The resulting genomic difference sequences constitute a group of nucleic acid sequences that are present in one genomic subtraction sample, but not in another. For example, subtraction between the genomes of a pathogenic strain of E. coli and a non-pathogenic strain of E. coli results in the isolation of a set of genomic difference sequences, each of which hybridizes to the nucleic acids of the pathogenic strain, but not to the nucleic acids of the non-pathogenic strain.

A number of different genomic subtraction methods have been applied to pairs of related strains to isolate pathogen-specific genomic difference sequences (for example, Mahairas et al., Journal of Bacteriology 178:1274-1282, 1996; Tinsley et al., Proc. Natl. Acad. Sci. U.S.A. 93:11109-11114, 1996). Such sequences have been used as diagnostic markers to identify and fingerprint other closely related strains (see, for example, Darrasse et al., Applied and Environmental Microbiology 60:298-306, 1994). Briefly, genomic subtraction is applied to the genomic DNA of two related strains and genomic difference sequences are isolated. A set of the genomic difference sequences is hybridized (each in a separate hybridization reaction) to the genomes of other strains from the same group. The subset of the genomic difference sequences that hybridizes to the genome varies from strain to strain, and thus constitutes an identifying fingerprint. Although this approach has been shown to be a powerful method for identifying closely related members of a biological group, it is too technically demanding, time consuming, and cumbersome to be implemented in a clinical setting. Furthermore, the genomic difference sequences in these experiments are usually derived from a single pathogenic strain, and therefore are only useful for typing very closely related strains of a single group. Thus, the prior art is incapable of exploiting genomic difference sequences for simultaneously testing numerous sequences from diverse organisms in a presentation-specific test.

It is also useful to identify an organism as a member of a larger biological grouping. For example, it may be important to determine whether an infection of the lower respiratory tract is due to any member of the species Bordetella pertussis. In this case one could, by nucleic acid hybridization, test for the presence of sequences that occur in all strains of this species, but do not occur in any other species. Such ID sequences, which distinguish members of one group from other groups, are called group-specific sequences.

Many of the most medically significant and diagnostically useful genetic variations are single-nucleotide polymorphisms (SNPs). For example, a single base-pair change in the globin gene is the cause of sickle-cell anemia. Single base-pair changes in the gene for RNA polymerase in Mycobacterium tuberculosis are the cause of resistance to rifampin, which is one of the most important antibiotics used to treat tuberculosis. Hybridization-based methods for detecting many SNPs at once have been developed, but these methods generally lack robustness due to the difficulty in discriminating between hybrids with exact matches and those with a single nucleotide mismatch (Gingeras et al., Genome Res. 8:435-438, 1998; Wan et al. Science 280:1077-1082, 1998). Some methods for genotyping SNPs only test for mutations at a single gene (Gingeras et al., 1998, supra). Other methods rely on multiplex PCR methodology, which suffers from irreproducibility.

Thus, there is a need for a method for genotyping many SNPs at once that uses robust hybridization and amplification methodologies.

Thus, to identify organisms, it is useful to test for the presence of ID sequences, which may include genomic difference sequences and/or group specific sequences. Testing for ID sequences, without culturing a medical sample, requires a method for detecting small numbers of genomes (e.g., 100-1000 genomes). Sensitive methods relying on nucleic acid amplification have been developed but, in general, as is described above regarding multiplex PCR, these methods can only be reliably applied to a very small number of sequences at once. Thus, the sensitive amplification-based methods that have been approved for clinical use test for only one or two pathogens at a time. These tests are much more expensive (often by a factor of about 100) than the standard microbiological tests performed in clinical laboratories. Consequently, commercial development of amplification-based assays has been limited to diagnostic tests for a small subset of organisms that cause common and severe infections and that cannot be easily grown in culture (e.g., HIV, Mycobacterium tuberculosis, and Chlamydia trachomatis). There is a need to extend the power and sensitivity of this technology to routine diagnostics.

Finally, it is often important to quantify a pathogen in a biological sample. For example, samples used to diagnose lower respiratory infections (e.g., pneumonia) are frequently contaminated with the normal commensal flora from the upper respiratory tract. Further compounding the diagnostic complications, many of the species that are harmless in the upper respiratory tract can be the cause of lower respiratory infections when they breach the respiratory system's normal defenses. In this case, knowledge of the numbers of organisms in the lower respiratory sample is important for differentiating between upper respiratory tract contamination and lower respiratory tract infection.

Quantitative analysis of pathogens in clinical samples is relatively straightforward if the organisms can be cultured. However, many medically important organisms are difficult or impossible to culture (e.g., most viruses, parasites, chlamydia, and anaeorbic bacteria). Furthermore, quantitative culture generally requires several days and may take more than a month for certain cases, such as culturing Mycobacterium tuberculosis, which causes tuberculosis. In a limited number of cases, quantitative data can be obtained by methods that do not require culture, such as immunological direct fluorescence assays. New molecular methods for quantitative analysis of pathogens, such as the quantitative polymerase chain reaction (PCR) have been very important in monitoring virus levels in AIDS patients. However, quantitative amplification methods are notoriously problematic to design correctly, can be irreproducible, and currently can only be applied to a single species at a time.

Thus, there is a need for a method that measures pathogen numbers in a biological or clinical sample. Such a method ideally would be rapid and general, i.e., it would not require culture and would quantify the numerous types of organisms that might be present in a sample.

In summary, a robust and sensitive identification method is needed that rapidly and accurately tests an uncultured sample for a large number of pathogen-specific sequences (genomic difference sequences and group-specific sequences and single-nucleotide polymorphisms) that are diagnostic of a diverse set of infectious agents that may cause a particular presentation (such as pneumonia). Such a test is also needed to provide medical and forensic information about the individual from which the sample is derived.

### SUMMARY OF THE INVENTION

The invention, in one aspect, provides a method, referred to as genomic profiling, to test an unknown biological sample simultaneously for the presence of nucleic acid sequences (including genomic difference sequences, group-specific sequences, and DNA polymorphisms) that are diagnostic of numerous (e.g., more than 5) different types of organisms. Genomic profiling represents a significant improvement over prior methods, as it (1) simultaneously scans a sample for the presence of a broad spectrum of organisms (e.g., viruses, bacteria, fungi, parasites, and human cells), (5) provides high resolution genetic identification information, (3) tests for specific mutations (such as those underlying genetic disease or antibiotic resistance), (4) offers speed and simplicity, (5) does not require a limiting and time consuming culture step. (6) makes it possible to sensitively test a complex “raw” sample for a much larger number of diagnostic sequences than was previously possible, (7) achieves robustness by incorporating a high degree of redundancy and internal controls, and (8) provides a means for quantifying the number of target organisms in a sample. This combination of attributes enables a new type of comprehensive, presentation-specific diagnostic test for infectious disease. For example, genomic profiling makes it feasible to offer to an individual suffering from respiratory symptoms a single test that simultaneously and rapidly scans for the presence of all common respiratory pathogens, including such diverse pathogens as bacteria, viruses, and fungi.

Accordingly, the invention features a method for obtaining genetic information from a biological sample potentially containing target nucleic acid molecules by: (a) providing nucleic acid molecules that are (i) target nucleic acid molecules in the sample, or (ii) probes that hybridize to target nucleic acid molecules in the sample, or (iii) amplification products of (i) or (ii), or (iv) a genomic representation of (i); and (b) detecting target nucleic acid molecules by contacting or comparing the nucleic acid molecules of (a) with a detection ensemble that has a minimum genomic derivation of greater than five (e.g., greater than eleven), and that includes detection sequences that can detect target nucleic acid molecules. This method can also include the step of (c) identifying nucleic acid molecules detected in step (b).

In preferred embodiments, the nucleic acid molecules of step (a) are not immobilized as size-fractionated fragments in a matrix or on a solid support prior to step (a); the amplification step is carried out using fewer than four pairs (e.g., a single pair) of amplification sequences, to yield, if target nucleic acid molecules are present in the sample, amplification products; and the method is used to quantify a target organism in the biological sample by in situ hybridization.

A preferred format of the method, exemplified in Example 2, below, involves, prior to step (a), the step of hybridizing nucleic acid molecules of the sample, simultaneously, with an ensemble of ID probes to yield the probes of step (a)(ii), above.

Preferably, the probes of step (a)(ii) include (i) a first region capable of hybridizing to a target nucleic acid molecule, and (ii) amplification sequences. Hybridization can be carried such that all of the nucleic molecules in step (a) are in the liquid phase or, alternatively, such that at least some of the nucleic acid molecules in step (a) are fixed to a solid support. Additionally, at least some of the nucleic acid molecules of step (a) can include one or more oligonucleotide tags.

At least some of the probes of step (a)(ii) can include (i) two or more oligonucleotides that can be ligated to one another upon hybridization to a target nucleic acid molecule, and (ii) amplification sequences.

In another embodiment, at least 50% of the probes of the ensemble of nucleic acid probes are capable of hybridizing to pre-determined genomic difference sequences that are potentially present in the sample or in a genomic representation of the sample.

In a preferred embodiment, the oligonucleotides that can be ligated to one another, as are mentioned above, are SNP probes. At least some of the SNP probes can include a tag sequence that can hybridize to one tag sequence in a detection ensemble that contains an ensemble of tag sequences. The minimum genomic derivation of the detection ensemble in these embodiments can be, for example, greater than twenty (e.g., greater than fifty).

In some preferred embodiments, the detection sequences of the detection ensemble are arrayed as spots in two dimensions or as parallel stripes on a solid support.

In other embodiments, the amplification products of step (a)(iv) are generated by amplification of target nucleic acid molecules of step (a)(i) using no more than four pairs of amplification sequences, e.g., amplification sequences that direct the amplification of sequences lying between Alu repeats using Alu-specific primers. In these embodiments, the detection ensemble of (b) can include ID sites that are congruent to ID probes potentially amplified in step (a)(iv).

The invention is useful for detecting and quantifying any type of organism. For example, in one preferred embodiment, the ensemble of ID probes includes probes that hybridize to at least two different nucleic acid molecules from each of at least ten different viruses, each of which belongs to a different genus.

The invention is useful in connection with many types of biological samples, including clinical samples. In one example, the biological sample is a sample from a human gastrointestinal tract, and the genetic information obtained using the method of the invention is the identification of nucleic acid molecules in the sample from six or more of the organisms Escherichia coli, Salmonella, Shigella, Yersinia enterocolitica, Vibrio cholera, Campylobacter fecalis, Clostridium difficile, Rotavirus, Norwalk virus, Astrovirus, Adenovirus, Coronavirus, Giardia lamblia, Entamoeba histolytica, Blastocystis hominis, Cryptosporidium, Microsporidium, Necator americanus, Ascaris lumbricoides, Trichuris trichiura, Enterobius vermicularis, Strongyloides stercoralis, Opsthorchis viverrini, Clonorchis sinensis, and Hymenoplepis nana.

In another embodiment, the biological sample is a respiratory tract sample, and the genetic information is the identification of nucleic acid molecules from six or more of the organisms Cornybacterium diphtheriae, Mycobacterium tuberculosis, Mycoplasma pneumoniae, Chlamydia trachomatis, Chlamydia pneumoniae, Bordetella pertussis, Legionella spp. Nocardia spp., Streptococcus pneunoniae, Haemophilus influenzae, Chlamydia psittaci, Pseudomonas aeruginosa, Staphylococcus aureus, Histoplasma capsulatum, Coccidoides immitis, Cryptococcus neoformans, Blastomyces dermatitidis, Pneumocystis carinii, Respiratory Syncytial virus, Adenovirus, Herpes Simplex virus, Influenza virus, Parainfluenza virus, and Rhinovirus.

Another biological sample that can be tested according to the invention is a blood sample, in which nucleic acid molecules are identified from at least six of the organisms Coagulase-negative staphylococci, Staphylococcus aureus, Viridans streptococci, Enterococcus spp., Beta-hemolytic streptococci, Streptococcus pneumoniae, Escherichia spp., Klebsiella spp., Pseudomonas spp., Enterbater spp., Proteus spp., Bacteroides spp., Clostridium spp., Pseudomonas aueruginosa, Cornybacterium spp., Plasmodium spp., Leishmania donovani, Toxoplasma spp., Microfilariae, Fungi, Histoplasma capsulatum, Coccidoides immitis, Cryptococcus neoformans, Candida spp., HIV, Herpes Simplex virus, Hepatitis C virus, Hepatitis B virus, Cytomegalovirus, and Epstein-Barr virus.

The invention can also be used to identify nucleic acid molecules in any type of biological sample, in which the identified nucleic acid molecules are of six or more of the organisms Coxsakievirus A, Herpes Simplex virus, St. Louis Encephalitis virus, Epstein-Barr virus, Myxovirus, JC virus, Coxsakievirus B, Togavirus, Measles virus, a Hepatitis virus, Paramyxovirus, Echovirus, Bunyavirus, Cytomegalovirus, Varicella-Zoster virus, HIV, Mumps virus, Equine Encephalitis virus, Lymphocytic Choriomeningitis virus, Rabies virus, and BK virus.

The invention also features a method for obtaining genetic information from a biological sample potentially including target nucleic acid molecules by (a) providing an ensemble of nucleic acid probes having a minimum genomic derivation of greater than five; (b) contacting the ensemble of probes, simultaneously, with nucleic acid molecules of the sample; (c) detecting hybridization between the probes and any target nucleic acid molecules of the sample; and (d) identifying nucleic acid molecules detected in step (c).

Also featured in the invention is a kit for obtaining genetic information from a biological sample, which includes: (a) a plurality of ID probes and/or SNP probes; and (b) a detection ensemble including detection sequences that are congruent with probes of (a) and having a minimum genomic derivation of greater than five (e.g., greater than eleven).

In preferred embodiments, the probes of (a) include more than ten (e.g., more than fifty or more than two hundred and fifty) different amplifiable probes; at least 50% of the probes of (a) include genomic difference sequences from at least three different species; the probes of (a) include more than five families of amplifiable probes; and the probes of (a) are specific for at least two distinct taxa, two different species, two different genera, or two different kingdoms.

In other preferred embodiments, the probes of (a) include probes that include: (i) two or more oligonucleotides that can be ligated to one another upon hybridization to an ID sequence of a target nucleic acid molecules, and (ii) amplification sequences.

In other embodiments, the probes of (a) and/or the detection sequences of (b) are physically attached to distinct locations on a solid support. In these embodiments, the detection sequences of the detection ensemble that detect (i) members of a taxonomic group and (ii) closely related taxonomic groups can be positioned adjacent to one another on the support.

The invention also features a kit for obtaining genetic information from a biological sample, which includes: (a) a plurality of nucleic acid primers (e.g., Alu-specific primers) that are capable of priming the amplification of DNA sequences flanked by repetitive sequences (e.g., human Alu repeats) in target genomic DNA in a biological sample to yield ID probes; and (b) a detection ensemble including detection sequences that are congruent with ID probes potentially amplified using the primers of (a), the detection ensemble having a minimum genormic derivation of greater than five (e.g., greater than twenty).

Also included in the invention is an ensemble of ID probes that can be amplified using fewer than four pairs of amplification sequences and that includes more than three (e.g., more than ten or more than twenty five) families of ID probes and more than ten (e.g., more than fifty or more than two hundred and fifty) different ID probes.

In preferred embodiments, more than two of the families of amplifiable probes are specific for non-overlapping taxa, different species, different genera, or different kingdoms. At least 50% of the probes can include genomic difference sequences from at least three different species.

In other preferred embodiments, the probes of (a) include probes that include: (i) two or more oligonucleotides that can be ligated to one another upon hybridization to an ID sequence of a target nucleic acid molecule, and (ii) amplification sequences.

In other preferred embodiments, the detection sequences included in the detection ensemble that detect (i) members of a taxonomic group and (ii) closely related taxonomic groups are positioned adjacent to one another on a support.

The procedures and reagents used in the invention are general, i.e., a single set of reagents can be used to identify many different types of organisms. The tests are rapid, and can simply incorporate positive and negative internal controls. The methods of the invention can generate high-resolution genetic fingerprints, identifying strains that are indistinguishable by conventional methods. The methods are amenable to automated formats, and can be carried out without extensive training of personnel.

The invention has a wide range of applications, including typing microorganisms (e.g., bacteria, fungi, and protozoa); determining the genotype of higher organisms (including humans); and, in epidemiology, monitoring infection outbreaks in hospitals and geographically remote regions. The methods of the invention also have utility in environmental testing, agriculture, both for breeding and analysis of livestock, and in plant typing, e.g., in the seed industry. Human forensics represents yet another application of the invention.

A critical feature of the invention lies in its ability to test for, in one assay, an ensemble of ID sequences that are useful for identifying organisms in a complex biological sample. The set of ID sequences comprise numerous genomic difference sequences, which distinguish members within a taxonomic group (e.g., different E. coli strains) and numerous group-specific sequences, which distinguish between different taxonomic groups (e.g., different species or genera). Each ensemble thus can include a very large array of different ID sequences, all of which can be used simultaneously in one rapid, non gel-based assay. The rapidity of the tests is enhanced by the fact that culturing of the samples is not required.

Other features and advantages of the invention will be apparent from the following detailed description, the drawings, and the claims.

Definitions

By a “genome” is meant the nucleic acid molecules in an organism that are the ultimate source of heritable genetic information of the organism. For most organisms, a genome consists primarily of chromosomal DNA, but it can also include plasmids, mitochondrial DNA, and so on. For some organisms, such as RNA viruses, a genome consists of RNA.

By “nucleic acid” is meant DNA, RNA, or other related compositions of matter that can include substitution of similar moieties. For example, nucleic acids can include bases that are not found in DNA or RNA, including, but not limited to, xanthine, inosine, uracil in DNA, thymine in RNA, hypoxanthine, and so on. Nucleic acids can also include chemical modifications of phosphate or sugar moieties, which can be introduced to improve stability, resistance to enzymatic degradation, or some other useful property.

By “oligonucleotide” or “oligonucleotide sequence” is meant a nucleic acid of length 6 to 150 bases. Oligonucleotides are generally, but not necessarily, synthesized in vitro. A segment of nucleic acid that is 6 to 150 bases and that is a subsequence of a larger sequence may also be referred to as an oligonucleotide sequence.

By “target sequence” or “target nucleic acid sequence” is meant a nucleic acid sequence that a probe is designed to detect. For an ID probe, the target sequence might be an ID site in an ID sequence.

For a SNP probe the target sequence might be a single-nucleotide polymorphism.

By “target organism” or “target group” is meant a type of organism or biological group (taxon) that a diagnostic test is designed to detect.

By “hybridization” is meant non-covalent binding of nucleic acid molecules mediated by hydrogen bonding of pairs of bases.

By “meaningful hybridization” is meant the hybridization, resulting in detection of a signal, of a probe molecule or molecules with the nucleic acid sequence that the probe is designed to detect.

By “comparative hybridization conditions” is meant the conditions used to distinguish species from each other as recommended by the International Committee on Systematic Bacteriology (Wayne et al. Internat. J. System. Bacteriol. 37:463-464, 1987). The comparative hybridization conditions referred to herein are those employed by Hartford et al (Int. J. Syst. Bacteriol. 43:26-31, 1993.

By “subtractive hybridization conditions” is meant conditions that are equivalent in stringency to the stringency of a reaction carried out at 65° C. in a buffer comprised of 10 mM EPPS, pH 8.0, and 1 M NaCl.

By a nucleic acid sequence, nucleic acid molecule, oligonucleotide, or probe that “is found in,” “is present in”, “occurs in,” “corresponds to,” “hybridizes to,” or “is in” another nucleic acid sequence, nucleic acid molecule, oligonucleotide, probe, or genome, is meant a sequence, oligonucleotide, or probe that can form a hybrid with another sequence, oligonucleotide, probe or genome that has a melting temperature (Tₘ) that is less than 20° C. (for sequences of greater than 30 bp), 12° C. (for sequences of 15 to 30 bp), or 8° C. (for sequences of 8 to 14 bp) below the Tₘ of a double-stranded DNA fragment composed of the shorter of the two nucleic acid molecules being compared and its exact complement in a buffer comprised of 10 mM EPPS, pH 8.0, and 1 M NaCl. By a nucleic acid sequence, nucleic acid molecule, oligonucleotide, or probe that “is absent in” another nucleic acid sequence, nucleic acid molecule, oligonucleotide, probe, or genome is meant a nucleic acid sequence, nucleic acid molecule, oligonucleotide, or probe that is not found in another nucleic acid sequence, nucleic acid molecule, oligonucleotide, probe, or genome.

By “ID sequence” or “identification sequence” is meant a nucleic acid sequence that is diagnostic of a particular organisms or group of organisms when its presence is assayed in a genome or enriched genome (see below) by hybridization using the length-specific melting temperature criteria described in the previous definition, ID sequences correspond to sequences in a genome or enriched genome that are ≧30 bp long and which are useful for distinguishing one type of organism from another. Genomic difference sequences can be used as ID sequences, for example, when it is important to distinguish members of a closely related group from each other. “Group-specific sequences” are a type of ID sequence that is useful for distinguishing all members of a group from other groups.

By “genomic difference sequence(s)” is meant a nucleic acid sequence or a collection of nucleic acid sequences that are found in the genome (or enriched genome) of one organism, but not in a closely related organism. Genomic difference sequences can be found by hybridization/subtraction techniques, by comparison of genome sequences using a computer, or by any of a variety of other techniques. The organisms whose genomes (or enriched genomes) are being compared must be “closely related.” A pair of organisms is considered “closely related” if they are members of the same genus or if their genomes fulfill the following specific hybridization criteria (note that comparative hybridization is recommended for establishing relatedness by the International Committee on Systematic Bacteriology (Wayne et al. 1987, supra)). A pair of organisms is considered “closely related” if more than 70% of their genomic DNA fragments (or genomic cDNA fragments in the case of viruses with RNA genomes) can hybridize with each other under comparative hybridization conditions using the method described by Hartford et al. (1993, supra). Genomic difference sequences are ≧30 bp in length. An example of a genomic difference sequence is a DNA fragment that occurs in one pathogenic strain of E. coli O157:H7, but that does not occur in another pathogenic strain of E. coli O157:H7.

By “group specific sequence(s)” is meant a nucleic acid sequence or a collection of nucleic acid sequences that is, by hybridization under comparative hybridization conditions, characteristic of the genomes of organisms in one phylogenetic group, but not of another taxon or phylogenetic group. Group-specific sequences are ≧30 bp in length. For example, a fragment that occurs in more than 99% of isolates in the E. coli O157:H7 group, but that is absent in more than 99% Salmonella isolates, is a group-specific sequence. Similarly, a fragment that occurs (as defined by hybridization under comparative conditions) in more than 99% of rotavirus isolates, but is absent in more than 99% of human immunodeficiency virus isolates, is a group-specific sequence. Group-specific sequences can be used to identify lower level taxonomic groups, such as subspecies or members of an interbreeding population (such as humans) that are related by descent. Note that, for diagnostic purposes, group-specific sequences are most useful when they occur in one taxonomic group, but not in a sister group at a similar taxonomic level.

An example of a group-specific sequence is one that is found in essentially all isolates of Salmonella enterica serotype Typhimurium, but that is found in essentially no isolates of Salmonella enterica serotype Paratyphi B (see FIG. 6). Note that group-specific sequences can also be genomic difference sequences (that is, the set of group-specific sequences overlaps with the set of genomic difference sequences). For example, a sequence that is in all E. coli O157:H7 strains, but that is in not found in non-O157:H7 strains of E. coli, is both a genomic difference sequence and a group-specific sequence.

By “conserved sequence” is meant a nucleic acid sequence or a collection of nucleic acid sequences that, by hybridization criteria, is characteristic of the genomes of organisms spanning multiple independent taxonomic groups at the same taxonomic level. Conserved sequences are ≧30 bp in length. Thus, the sequences of many fragments of the gene encoding human RNA polymerase are conserved sequences, as they can hybridize to the chimpanzee genome under comparative hybridization conditions. Conserved sequences are not useful for differentiating members of the groups harboring the conserved sequences.

By an “ID probe” is meant an oligonucleotide or a pair or a set of oligonucleotides that is used to hybridize to an ID sequence in a biological sample. To hybridize, a portion of the probe oligonucleotide must be capable of base-pairing with the corresponding ID sequence. This portion of the probe is typically between 8 and 120 bases in length, ID probes can also have other portions including amplification sites (for example, sequences that correspond to primer binding sites for PCR amplification) and sequences that serve as tags during detection (see below).

By a “genomic difference probe” is meant an ID probe that corresponds to, i.e., hybridizes to, a genomic difference sequence.

By a “group-specific probe” is meant an ID probe that corresponds to, i.e., hybridizes to, a genomic difference sequence.

By an “ID probe site” or “probe site” is meant the part of an ID sequence that corresponds in sequence to an ID probe.

By a “family of ID sequences” is meant a set of ID sequences comprising 2 or more members that can hybridize to the genome of a single (non-recombinant) organism (under comparative hybridization conditions). At least 2 of the ID sequences in the family must map further than 3,000 base pairs apart in the genome in which they naturally and typically occur. A family of ID sequences may comprise a combination of group specific sequences and genomic difference sequences, may comprise only group-specific sequences, or may comprise only genomic difference sequences.

Consider, for example, a family of ID sequences that is useful for tracking outbreaks of infectious E. coli O157:H7. This family of ID sequences can include all of the following types of diagnostically useful ID sequences: multiple group-specific sequences that are common to and limited to all members of the species E. coli; multiple group-specific sequences that are common to and limited to all members of the phylogenetic group containing only E coli O157:H7 strains; multiple group-specific sequences that are common to and limited to all members of the phylogenetic group containing only E. coli O157:H7 found by multienzyme electrophoretic analysis to have electrophoretic type 3 (DEC3 group; Whittam et al, Infect. Immun. 61:1619-1629, 1993); and multiple genomic difference sequences that are present in the E. coli O157:H7 reference strain DEC3B, but that are not present in the E. coli O157:H7 reference strain DEC4C.

Note that in the above example the family of ID sequences can all be hybridized under comparative hybridization conditions to the genome of a single organism: E. coli O157:H7 reference strain DEC3B. This is a defining aspect of the expression “family of ID sequences.”

By a “family of oligonucleotides” or a “family of probes” is meant a collection of oligonucleotides or probes corresponding to a family of ID sequences. All oligonucleotide or probe sequences in a family of oligonucleotides or probes correspond to all or part of the sequences of members of a particular of family ID sequences.

By “polymorphism probe” or “single-nucleotide polymorphism probe,” or “SNP probe” is meant a set of oligonucleotides that, when hybridized to a genome, abut at a polymorphic site and have sequences that lead to precise base-pairing at that site for one particular genomic sequence that occurs at the site. A set of such oligonucleotides, when hybridized adjacently to a genome, can be ligated to each other only when the allele, or genotype, at the targeted site matches the abutting sequences of the oligonucleotides of the polymorphism probe. The structure and use of SNP probes is illustrated in FIG. 10. Generally, a group of polymorphism probes is synthesized that correspond to each allele at a particular site. Polymorphism probes can comprise the same moieties as can ID probes (e.g., amplification sites and tags). An ensemble of polymorphism probes with tag sequences is useful for generating enriched genomic samples containing differences that can be detected by hybridization to a detection ensemble comprising an ensemble of tags.

A “family” of polymorphism probes, or “single nucleotide polymorphism probes” or “SNP probes,” is defined analogously to a family of ID sequences and ID probes, except in this case correspondence between a probe and genomic DNA rests on the ability of pairs of probe-halves to hybridize to and precisely abut at a polymorphic genomic site (e.g., a single base-pair polymorphism) rather than being based on the hybridization criteria used for ID sequences (see FIG. 10). For the purposes of defining a family of SNP probes, only one allele being tested by each SNP probe is considered. Only the SNP allele being tested by a particular SNP probe that has the smallest allelic frequency is considered. This allele is defined as the “most infrequent SNP allele target”. “Allelic frequency” is defined in a population of a species for a particular allele at a particular locus in the genome. The allelic frequency is the fraction of all alleles at the locus in the population that is represented by a particular allele (King, et al., A dictionary of genetics (Oxford University Press, New York, 1990). The population sample used to determine allelic frequency must include at least 100 (non-clonally related) individuals. A family of SNP probes is a set of SNP probes whose most infrequent SNP allele targets all occur in the genome of a single individual.

By a “tag” or “tag sequence” is meant a non-biological oligonucleotide sequence that may be incorporated within a larger oligonucleotide or probe. Tag sequences are useful as detection sequences. A tag sequence in a detection array can be used, for example, to detect, by hybridization, a (complementary) tag sequence in an amplified probe. Tag sequences can be used to distinguish probes from one another by hybridization in cases where different diagnostic sequences might not otherwise be distinguishable by hybridization (e.g., SNP probes; see below).

Similarly, by a “family of tag sequences” or “family of tags” is meant a set of tag sequences that corresponds to a family of probes. For example, in example 5, below, an ensemble of polymorphism or SNP probes is hybridized with a human genomic DNA sample. The subset of the ensemble of SNP probes that can be ligated and amplified is a family of SNP probes. This family is defined analogously to a family of ID probes, in that a family of SNP probes corresponds to the genotype of a single human individual. A family of tag sequences is contained by the family of SNP probes (SNP probes are generally constructed with an identifying tag sequence). Thus, the family of SNP probes is congruent to the family of tag sequences and can be identified by hybridizing to the congruent family of tag sequences in a detection ensemble.

By sets of sequences that are “congruent” is meant that there is a one to one correspondence between elements of the sets. For example, consider an ensemble of ID probes that is congruent to an ensemble of ID sequences. Each ID probe contains an ID site that lies within an ID sequence and every ID sequence corresponds to an ID probe. Or, consider a detection ensemble made up of an ensemble of tags that is congruent to an ensemble of polymorphic probes. Each tag in the detection ensemble corresponds to a tag in one of the polymorphism probes in the ensemble of polymorphism probes. Similarly, a family of tag sequences can be congruent to a family of polymorphism probes.

By “minimum genomic derivation” is meant the minimum number of distinct genomes (or the minimum number of distinct genomic representations) to which a set of sequences, probes, oligonucleotides, or tags can be hybridized. For example, the minimum genomic derivation of a set of ID sequences is equivalent to the minimum number of families that can be constructed from a set of ID sequences. So, for example, the minimum genomic derivation is one for a set of ID sequences, each of which corresponds to a protein-encoding segment of a different human gene, since the entire set of sequences could hybridize to the genome of a single human. As another example, consider a set of sequences consisting of a pair of group-specific adenovirus sequences and a pair of group-specific respiratory syncytial virus sequences. The minimum genomic derivation of such a set is 2, since the sequences of 2 genomes, adenovirus and respiratory syncytial virus, are the minimum number of genomes that are sufficient to hybridize to all 4 sequences under comparative hybridization conditions. The set of 4 ID sequences constitutes 2 families of ID sequences, as long as each pair of viral ID sequences is separated by ≧3000 bp in the genome of origin (see definition of “family” above).

It is also helpful to consider a more complicated example, illustrated in Table 1, of a set of ID sequences that can be used to test a patient with acute gastrointestinal illness for the presence of certain pathogens. Note that the group of sequences in each box in Table 1 can hybridize to the genomic DNA of a single individual. (There are 9 such boxes in Table 1.) Also, note that it is impossible to hybridize all of the sequences contained in the 9 boxes in Table 1 to the genomic DNA of fewer than 9 individuals. Thus, the minimum genomic derivation of the set of ID sequences in Table 1 is 9.

TABLE 1
An ensemble of ID sequences with a minimum genomic derivation of 9.
Each box in the table encloses a “family” of ID sequences
(i.e., a set of sequences that can hybridize to a single genome).

E. coli O157:H7 genomic difference sequence 2 (present in
E. coli O157:H7 strain X but not in E. coli O157:H7 strain Y)
E. coli O157:H7 group-specific sequence A
E. coli O157:H7 group-specific sequence B
E. coli group-specific sequence A
E. coli group-specific sequence B
E. coli O157:H7 genomic difference sequence 3 (present in
E. coli O157:H7 strain Y but not in E. coli O157:H7 strain X)
E. coli O157:H7 genomic difference sequence 4 (present in
E. coli O157:H7 strain Y but not in E. coli O157:H7 strain X)
E. coli O157:H7 group-specific sequence A
E. coli O157:H7 group-specific sequence B
E. coli group-specific sequence A
E. coli group-specific sequence B
E. coli O55:H6 genomic difference sequence (present in one
E. coli O55:H6 strain but not in another E. coli O55:H6 strain)
E. coli group-specific sequence A
Salmonella enterica serotype Typhimurium genomic difference sequence 1
(present in one Salmonella enterica serotype Typhimurium strain but not
in another Salmonella enterica serotype Typhimurium strain)
Salmonella enterica serotype Typhimurium genomic difference sequence 2
(present in one Salmonella enterica serotype Typhimurium strain but not
in a Salmonella enterica serotype Paratyphi B strain)
Salmonella enterica group-specific sequence
Salmonella enterica serotype Typhimurium group-specific sequence
Salmonella enterica serotype Paratyphi B genomic difference sequence 1
(present in one Salmonella enterica serotype Typhimurium
strain but not in another Salmonella enterica serotype Paratyphi B strain)
Salmonella enterica serotype Paratyphi B genomic difference sequence 2
(present in one Salmonella enterica serotype Typhimurium strain but
not in a Salmonella enterica serotype Typhimurium strain)
Salmonella enterica group-specific sequence
Salmonella enterica serotype Paratyphi B group-specific sequence
Campylobacter fecalis genomic difference sequence 1 (present in
Campylobacter fecalis strain X but not in Campylobacter fecalis
strain Y)
Campylobacter fecalis genomic difference sequence 2 (present in
Campylobacter fecalis strain X but not in Campylobacter fecalis
strain Z)
Rotavirus group-specific sequence 1
Rotavirus group-specific sequence 2
Rotaviris group-specific sequence 3
Norwalk virus group-specific sequence 1
Norwalk virus group-specific sequence 2
Norwalk virus group-specific sequence 3
Giardia lamblia genomic difference sequence 1
Giardia lamblia genomic difference sequence 2

The definition of minimum genomic derivation as applied to ensembles of SNP probes and ensembles of tag sequences is defined as follows. An ensemble of SNP probes consists of multiple families of SNP probes, and each family of SNP probes corresponds to the genotype of a single individual. However, as opposed to ensembles of ID sequences, an ensemble of SNP probes generally has a minimum genomic derivation of one. This is because SNP probes can generally hybridize to any genome of the target species with no more than a single base-pair mismatch.

Now, consider an ensemble of human SNP probes, each of which includes a unique tag sequence moiety. Also, consider a detection array comprising an ensemble of tags congruent to the tag sequences in the ensemble of SNP probes. The ensemble of SNP probes generally has a minimum genomic derivation of one, since all members can hybridize to any particular human genome. However, note that, in contrast, the congruent ensemble of tags may have a large minimum genomic derivation. To understand this apparent paradox, it helps to realize that the ensemble of SNP probes is composed of families of SNP probes, each of which corresponds to the genotype of a single individual. The set of tag sequences in the family of SNP probes is a congruent family of tag sequences. The congruent family of tag sequences in the detection array can hybridize to such a family of SNP probes. However, the other tag sequences in the ensemble of tags cannot hybridize to that family of SNP probes. So, the minimum genomic derivation of an ensemble of tag sequences that is congruent to an ensemble of SNP probes is equal to the number of families in the ensemble of SNP probes—even though the minimum genomic derivation of the ensemble of SNP probes itself is 1.

The definition of minimum genomic derivation as applied to an ensemble of tags depends on the following definitions. Recall the definition of “the most infrequent SNP allele target” for a particular SNP probe (see definition of “family of SNP probes” above). I define “the most frequent SNP allele target” in an analogous manner. Thus, for the alleles tested for by a particular SNP probe, one allele is determined to be the least common within a species and one allele is determined to be the most common. The “average allelic frequency” of a SNP probe is defined to be the average of the allelic frequencies of the most frequent SNP allele target and the least frequent SNP allele target. For example, if the alleles that can be detected by a SNP probe occur at frequencies 0.85, 0.06 and 0.002, the average allelic frequency is 0.426 (i.e., (0.85+0.002)÷2)) The “product of the average allelic frequencies” (P) is defined as the product of the allelic frequencies for all of the SNPs in the SNP ensemble. So, for example, consider a hypothetical test in which SNP probes are used to test for 36 human disease mutations each of which occurs with an allele frequency of 0.001 and each of which is associated with a normal allele that occurs with an allelic frequency of 0.999. For each of the 36 SNPs the average allelic frequency is 0.5 (i.e., (0.001+0.999)÷2)). The product of the average allelic frequencies (P) is therefore 0.5³⁶=1.46×10⁻¹¹. (Note that for an actual ensemble of SNP probes the value of the allelic frequencies and average allelic frequencies will vary from probe to probe. Also, note that the allelic frequencies for a SNP probe need not add up to 1.0, as not all of the alleles that occur need be assayed by the SNP probe).

Since, in practice, it can be difficult to determine the minimum number of families comprising an set of SNP probes for a particular species, I define the minimum genomic derivation for an ensemble of tags that is congruent to an ensemble of SNP probes in the following way. The minimum genomic derivation of an ensemble of tags is defined as (10⁻¹⁰)(P)⁻¹, where P is the product of the average allelic frequencies. Thus, in the previous example, the minimum genomic derivation of the ensemble of tags congruent to the ensemble of human disease mutation SNP probes is (10⁻¹⁰)(1.46×10⁻¹¹)⁻=6.9. In contrast, as explained above, the minimum genomic derivation of the congruent ensemble of SNP probes is one.

I offer the following example to give a sense of the biological rationale for the definition of the minimum genomic derivation for a set of tags congruent to a set of SNP probes. Consider a set of 33 tags that is congruent to a set of unlinked human SNP probes each of which detects two alleles both of which have an allelic frequency of 0.5. The minimum genonic derivation of this set of tags is (10⁻¹⁰)(P)⁻¹⁼(10⁻¹¹)(0.5³³)⁻¹=0.85, which is close to one. Note that the most probable genotype found would be an individual that is heterozygous at each of the 33 SNP loci (the probability of being heterozygous at such a locus is 0.5). The probability of finding an individual with the most probable genotype is 0.5³³=1.2×10⁻¹⁰. Such an individual would be expected to occur with a probability of a bit less than once in the total human population in the year 2000 (˜6×10⁹).

A detection ensemble can comprise detection sequences that are congruent to an ensemble of probes containing both ID probes and SNP probes (i.e., the detection ensemble has ID site sequences and tag sequences). The minimum genomic derivation of such an ensemble is the sum of the minimum genomic derivation of the ID sites plus the minimum genomic derivation of the tag sequences. If the ensemble of tags covers more than one species, the minimum genomic derivation of the ensemble is the sum of the minimum genomic derivations of the tags corresponding to each species.

By an “ensemble of ID sequences” is meant a set of ID sequences that corresponds to multiple families of ID sequences. That is, an ensemble of ID sequences has a minimum genomic derivation of more than 1. Furthermore, since each family is minimally composed of 2 (well-separated) ID sequences, an ensemble of ID sequences has a minimum membership of 4 ID sequences. A characteristic of an ensemble of ID sequences is that the genome of a single organism is not sufficient to give a positive hybridization signal with all the individual ID sequences. An ensemble of ID sequences is not necessarily physically isolated from samples. Rather, such an ensemble may be merely conceptualized to facilitate the design of ID probes for use in constructing a probe ensemble (see below). FIG. 1 diagrams an ensemble of ID sequences that has a minimum genomic derivation of 9 and that is described in Table 1.

By an “ensemble of ID oligonucleotides” or “ensemble of ID probes” is meant a collection of oligonucleotides or probes, each of which contains an oligonucleotide sequence that corresponds to all or a portion of an ID sequence in one particular ensemble of ID sequences. Such ensembles are designed to detect, by hybridization, nucleic acid sequences present in a sample that correspond to two or more distinct genomes (see below). Preferably, in an ensemble of probes, the sequences and/or concentrations of the probes in an aqueous solution are known.

By an or “ensemble of SNP probes” or “ensemble of single-nucleotide polymorphism probes” or “ensemble of polymorphism probes” is meant a set of SNP probes that comprises more than one family of SNP probes.

By an “ensemble of tag sequences” or “ensemble of tags” is meant a set of tag sequences that is congruent to an ensemble of probes. That is, each tag sequence in an ensemble of tag sequences is complementary to a tag sequence (or to the reverse complement of a tag sequence) in an ensemble of probes. Ensembles of tag sequences are useful in genomic profiling for converting single-nucleotide polymorphism genotypes (which are difficult to detect by hybridization) into robust hybridization genotypes (see example 5 below).

By an “ensemble” of some physical or chemical property is meant a set of values pertaining to said physical or chemical property that is congruent to an ensemble of nucleic acid sequences. For example, there is an ensemble of molecular weights matching one to one with the molecular weights of an ensemble of ID probes. Such an ensemble of molecular weights could be used as a detection ensemble, or detection array, to determine the identities of the elements of a sample-selected subset of an ID probe ensemble. The subset of ID probes could be analyzed by mass spectrometry and the observed molecular weights compared to the ensemble of molecular weights (i.e, the molecular weights of the original ensemble of ID probes).

By “detection ensemble” or an “ensemble of detection sequences” is meant a collection of sequences, referred to as “detection sequences,” all of which correspond to all or a portion of the members of an ensemble of sequences, probes, oligonucleotides, or tags (e.g., an ensemble of ID probes or of SNP probes). That is, a detection ensemble is congruent to an ensemble of sequences, probes, oligonucleotides, or tags. Such ensembles are designed to detect (usually, but not necessarily by hybridization) a diagnostically informative subset of an ensemble of ID probes, ID sequences, polymorphism probes, or other genomic representation containing diagnostically useful sequences. As is noted below, the components of a detection ensemble (i.e., detection sequences) may be positioned in a two-dimensional array, to facilitate identification of diagnostic probes (e.g., ID probes that have hybridized to ID sequences within the nucleic acid molecules of a sample). Alternatively, the elements of the detection ensemble may be contacted with diagnostic probes in liquid. Also as is noted below, ID probes that have hybridized to ID sequences within the nucleic acid molecules of a sample may be amplified before contact with a detection ensemble.

A detection ensemble can also be a set of values of a physical or chemical property that has a one to one correspondence with (i.e., that is congruent to) an ensemble of sequences, probes, oligonucleotides, or tags. For example, a list or array of molecular weights of the members of an ensemble of ID probes is one type of detection ensemble. Such a detection ensemble is useful for mass spectroscopic identification of a particular subset of the ensemble of ID probes. The molecular weights of a family of ID probes selected by a clinical sample can be determined using mass spectrometry. The molecular weights of the ID probe family are then compared to a detection ensemble of molecular weights (i.e., the molecular weights of the original unselected ensemble of ID probes). In this way, the selected ID probes are identified leading to, in turn, identification of the genomes in the clinical sample. Alternatively, as described in Example 3 below, a family of probes can be detected by hybridization to a detection ensemble of oligonucleotides. The probe-selected subset of detection oligonucleotides can then be identified by determining the molecular weights of the oligonucleotides and comparing to another detection ensemble: an array of molecular weights of the elements of the detection ensemble of oligonucleotides.

By a “two-dimensional detection array” is meant an ensemble of either ID sequences, ID oligonucleotides, ID probes, or detection sequences that have been positioned by a non-electrophoretic method to an essentially two-dimensional (i.e., planar) solid support, such as a nylon filter or a polylysine-coated glass slide.

By “genomic profiling assay” is meant certain methods of the invention.

By “genomic profiling fingerprint” or “fingerprint” is meant the subset of diagnostic sequences (e.g., ID probes or SNP probes) whose presence in a biological sample is inferred based upon the diagnostic probes that are amplified and detected by the genomic profiling assay.

By “taxon” (plural taxa) or “phylogenetic group,” is meant the collective members of a monophyletic group, that is a group of organismal types that descend from and include a common ancestral organismal type (either known or hypothesized). Note that for the purposes of this invention taxon is used in a general sense that does not imply any level of classification. Thus, for example, taxa are defined at the sub-species level and also at the level of genera, class, phylum, etc.

By “independent taxonomic groups” or “independent taxa” are meant taxa with non-overlapping membership. Thus, the bacterial genera Escherichia and Salmonella are independent taxa. However, the genus Escherichia and the taxonomic group consisting of Escherichia coli O157:H7 pathogens are not independent taxa, as all members of the pathogenic strain are also members of the genus.

By “taxonomic level” is meant the position of a taxon in the phylogenetic hierarchy. The terms isolate, ecotype, sub-species, species, genus, family, class, order, phylum, kingdom, and super-kingdom are examples of taxonomic levels.

By “kingdom” of living things is meant one of the following: viruses, bacteria, archaebacteria, fungi, protozoa, plants, and animals.

By “distinct genome” is meant a genome with a particular nucleic acid sequence that differs from those of all other genomes, except those of genetically identical organisms. Different organisms possessing distinct genomes can be unrelated or closely related. Clonal relatives, such as the genetically homogenous organisms within a bacterial colony, are said to possess the same distinct genome.

By “sample” is meant a collection of material from which nucleic acids are prepared and tested for the presence of particular nucleic acid sequences. A sample can be, for example, a sample of stool, urine, blood, or sputum, or other such samples that are routinely collected at hospitals. Alternatively, a sample can be a single colony of microorganisms growing in a petri dish. A sample can also be a human forensic sample, a food sample, an environmental sample, or pure nucleic acid.

By an “amplification methodology” or “amplification method” is meant a technique for linearly or exponentially increasing the copy number of a nucleic acid molecule. Examples of amplification methods include ligase chain reaction, PCR, ligation-dependent PCR, transcription-mediated amplification, strand-displacement amplification, self sustaining sequence replication, Qβ-replicase mediated amplification, rolling-circle amplification, and so on.

By “amplification products” are meant the nucleic acid molecules resulting from applying an amplification method.

By “amplification site” or “amplification sequence” is meant a region of a nucleic acid molecule that mediates or is required for replication by an amplification methodology. An example of a pair amplification sites is the pair of sites on a DNA fragment or chromosome to which oligonucleotide primers bind during specific priming in the PCR reaction. The promoter sequences for RNA polymerases, such as Qβ-replicase or phage T7 polymerase, that are used in certain amplification methods constitute another type of amplification site.

By “genomic subtraction” is meant a method that leads to the isolation of genomic difference sequences.

For example, hybridization methods in which a “+” DNA genomic difference sample (see below) is annealed to a “−” genomic difference sample and residual non-annealed “+” sequences are then isolated. An alternative example is the comparison of two sequence sets using a computer to yield sequences present in the first set and not the second. A sequence (30 bases in length) in the “+” sample is considered absent from the “−” sample if the sequence cannot hybridize to the “−” sample under subtractive conditions. That is, under subtractive conditions, the sequence cannot form a hybrid with a sequence in the “−” sample with a melting temperature (Tₘ) that is greater than 5° C. less than the temperature of the subtractive hybridization conditions. Hybridization can be experimentally determined or predicted based on known sequences.

By a “pair of genomic difference samples” is meant two sets of nucleic acid sequences, corresponding to genomic DNA or RNA, that are used to discover genomic difference sequences. For example, in a genomic subtraction experiment, the “+” and “−” DNA samples are the genomic difference samples. When comparing two genomes by computer analysis, each genome is a genomic difference sample. A genomic difference sample can be derived from a single organism or a group of organisms; can comprise amplified or unamplified nucleic acid, such as polymerase chain reaction (PCR)-amplified DNA; can be composed of fractionated nucleic acids, such as a size fraction or an amplified fraction; can be a deduced nucleic acid sequence, such as a computer representation of a sequence from a completely or almost completely sequenced genome; and can consist of RNA, DNA, or any other closely related nucleic acid molecule. A genomic difference sample is only meaningful if many, but not all, of the sequences in the “+” sample are also present in the “−” sample.

By an “enriched genome,” “enriched genomic fraction,” “enriched genomic difference sample,” or “genomic representation” is meant a genome, genomic fraction, or genomic difference sample that has undergone an enrichment procedure that generates a selected fraction of the original genome or genomic difference sample. For the purposes of genomic profiling, enriched genomes have two important attributes: (1) they offer robust hybridization-based diagnostics (compared to methods that detect SNPs by hybridization), and (2) enriched genomic fractions generated by amplification are an efficient way to generate material from small samples (such as forensic samples). For example, the source of a forensic hair sample can be identified by genomic profiling by testing for a large number of polymorphic sequences lying between Alu repeats in enriched genomes generated by Alu-PCR (see example 4). The genomic enrichment can be based on size fractionation, differential amplification (e.g., Alu-PCR or differential amplification of SNP probes), or any other fractionation method.

TABLE 2
Examples of genomic representations and their utility as
detection sequences.

| Representation of | Category of | Example of type of detection |
|---|---|---|
| genome | representation | sequence |
| Amplified size frac- | physical property | Restriction fragment length |
| tion of a restriction | (size) of restric- | polymorphism (RFLP), i.e., a |
| digested genomic | tion fragments | sequence that is in a size |
| DNA | | fraction in one strain but |
| | | absent in the same size |
| | | fraction in another strain |
| Amplification of | an amplified dif- | alu-morphs (sequences lying |
| sequences between | ferential ampli- | between alu repeats that |
| repeated sequences | fication depending | are amplifiable from one |
| | on arrangement of | chromosome but not from a |
| | repeats | homologous chromosome due |
| | | to polymorphism) |
| Amplification with | an amplified family | tags on the amplified SNPs |
| ensemble of SNP | of SNPs (i.e., SNPs | |
| probes | that represent the | |
| | genotype of one | |
| | individual | |
| Amplification of ID | an amplified family | Ensemble of ID sequences |
| probes that hybrid- | of ID probes | |
| ize to a sample | | |

### BRIEF DESCRIPTION OF THE DRAWINGS

FIG. 1 is a schematic illustration of an ensemble of ID sequences with a minimum genomic derivation of 9.

FIG. 2A is a schematic illustration of a phylogenetic tree showing the ancestral relationship of a hypothetical, but typical, group of strains including pathogenic (e.g., strain 1) and non-pathogenic (e.g., strain 8) variants.

FIG. 2B is a schematic illustration of a method of the invention, in which genomic subtraction using two organisms in a group of related strains (e.g., strains 1 and 8) yield genomic difference sequences that can be used for fingerprinting any strain within the group (e.g., strains 2-7).

FIG. 2C is a schematic illustration of a method of the invention, in which genomic difference sequences are generated by pooling genomic nucleic acid molecules from several organisms. For example, a “+” sample can be generated by pooling genomic nucleic acid molecules of several pathogens, and a “−” sample can be generated by pooling genomic nucleic acid molecules of several non-pathogens. The genomic difference sequences obtained by this subtraction experiment comprise sequences that occur in at least one of the pathogenic (“+”) strains but in none of the non-pathogenic (“−”) strains.

FIG. 3 is a schematic illustration of a binary ID probe that can be used in a method of invention. After hybridization to a chromosomal ID sequence, the left and right ID probe-halves are ligated to each other. Primers corresponding to primer site-L and primer site-R are then used to amplify the ligated product. The amplified ID probes product can be identified by subsequent hybridization to a detection array containing either the ID probe or the tag sequences (not shown in figure).

FIG. 4 is a schematic illustration of examples of different types of detection arrays.

FIG. 5 is a schematic illustration of a method of the invention in which a clinical sample is scanned for numerous pathogens by genomic profiling using sample-selection of ID probes. In this method, DNA from a sample is deposited onto a solid support, such as a nylon filter. Pairs of probe-halves are then hybridized to the bound sample DNA, and correctly hybridized probes are then ligated, eluted from the filter, and amplified for detection on a detection array.

FIG. 6 is a schematic illustration of a genomic subtraction strategy for obtaining genomic difference sequences from Salmonella enterica. In this strategy, the subspecies of S. enterica are divided into two subgroups, Group X and Group Y. Reciprocal subtractions are carried out to obtain a genomic difference sample for each of the groups.

FIG. 7A is a schematic illustration of part of the phylogenetic tree of the Escherichia coli group. Pathogens are colored black and non-pathogens are colored white.

FIG. 7B is a schematic illustration of a strategy for obtaining genomic difference sequences for E. coli O157:H7, in which genomic subtraction is carried out between E. coli O157:H7 (“+” genomic difference sample) and non-pathogenic strains (“−” genomic difference sample).

FIG. 7C is a schematic illustration of a strategy for obtaining genomic difference sequences for Shigella flexneri, in which genomic subtraction is carried out between Shigella flexneri (“+” genomic difference sample) and non-pathogenic strains (“−” genomic difference sample).

FIG. 8A is a schematic illustration of an ID probe (comprising a gapped circle probe and a gap probe) for use in rolling circle amplification.

FIG. 8B is a schematic illustration of a pair of primers (a biotinylated rolling circle primer and a biotinylated branching primer) for use in rolling circle amplification of the ligated rolling circle template.

FIG. 8C is a schematic illustration of hyperbranched rolling circle amplification carried out using the primers illustrated in FIG. 8B and the ligated rolling circle template.

FIG. 9A is a schematic illustration of a pair of biotinylated DNA capture probes, a pair of amplification probes, and a gap probe, each of which hybridizes to a ID sequence, as indicated.

FIG. 9B is a schematic illustration of amplification of a tripartite ligated probe using a pair of biotinylated primers.

FIG. 9C is a schematic illustration of hybridization between a gap probe sequence and an oligonucleotide for mass spectrometry detection.

FIG. 10 is a schematic illustration of SNP probe hybridization-selection, in which ligation and amplification depend on match at SNP site.

FIG. 11 is a schematic illustration of the common features of three general classes of genomic profiling methods of the invention.

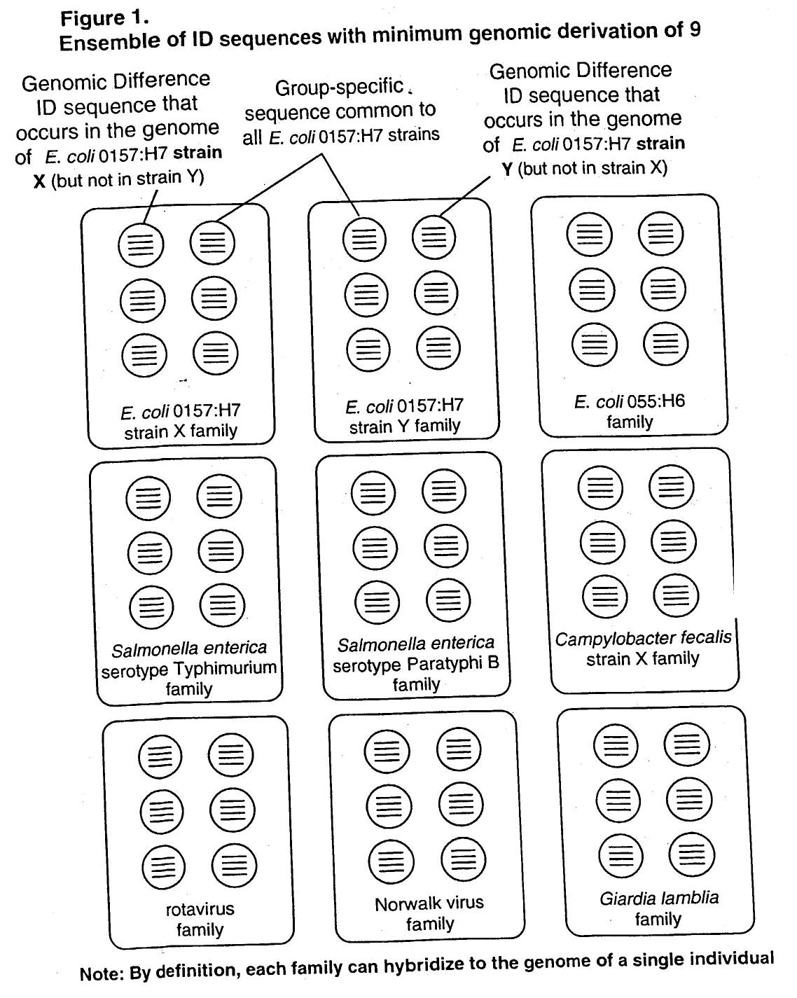

### DETAILED DESCRIPTION

Genomic profiling is a method for identifying or typing organisms that offers several significant advantages over the prior art. In medical diagnostics, the method, which is amenable to implementation in clinical diagnostic settings, offers therapeutic and epidemiological advantages. A complex biological sample can be simultaneously, rapidly, and sensitively scanned for the presence of a large number of pathogen-specific sequences. Genomic profiling generates high-resolution genetic fingerprints that allow it to be used to distinguish between very similar strains. This is important in distinguishing between a pathogen and a closely related non-pathogen, between similar pathogens involved in separate outbreaks of a disease, and between an antibiotic sensitive and resistant strain of the same pathogen. The ability of the invention to scan for many diagnostic sequences is important for applications that screen patients for numerous genetic markers and for applications in genetic identification.

Genomic profiling enables a new type of presentation-specific assay that tests a patient's sample for a comprehensive set of disease causing pathogens. For example, genomic profiling makes it feasible to offer to an individual suffering from respiratory symptoms a single test that rapidly scans for the presence of all common respiratory pathogens, including such diverse pathogens as bacteria, viruses, and fungi.

Current methods for typing organisms usually involve culturing the organisms, which requires time for the organisms to grow, requires diverse culture conditions, and can be infeasible in a hospital setting for many organisms, including some bacteria and most viruses and eukaryotic parasites. The new method allows results to be obtained in hours (rather than the days and sometimes weeks required by current methods), since it does not require culturing.

Other advantages of genomic profiling are that the method requires minimal processing of clinical samples, it generates fingerprints of previously uncharacterized organisms, positive and negative internal controls are simply implemented gel electrophoresis is unnecessary, and the method is amenable to automated formats.

Genomic profiling combines highly-parallel, hybridization-based screening with sensitive nucleic acid amplification methodology to allow identification of a broad range of organism types in a single assay. A single test can scan a biological sample for the presence of a useful class of DNA sequence polymorphisms, called ID sequences, ID sequences are nucleic acid sequences that are specific to the genomes of organisms within a particular group. A single test can also simultaneously scan for numerous single-nucleotide polymorphisms (SNPs), another type of genomic variation. Genomic profiling can, in addition, test for mixtures of ID sequences and SNPs in a single test.

Two categories of ID sequences are useful for identifying organisms: group-specific sequences and genomic difference sequences, ID sequences that are present in all members of a related group of organisms are called group-specific sequences. Group-specific sequences are useful for determining if a member of a certain group is present in a biological sample. For example, the presence of an HIV group-specific sequence indicates the presence of a virus in the HIV group. Group-specific sequences can be isolated by computer comparisons of genomic databases or by molecular methods for isolating conserved sequences such as coincidence cloning.

ID sequences that are present in only some members of a group of related organisms are called genomic difference sequences. Sets of genomic difference sequences are particularly useful for obtaining high resolution fingerprints of organisms. Thus, this type of ID sequence facilitates distinguishing one member of a group from another member of a group. Fingerprinting organisms is important for epidemiology, forensics, and for rapidly determining whether a bacterium is likely to be resistant to certain antibiotics. Genomic difference sequences can be prepared, for example, by carrying out a subtractive hybridization procedure with the genomes of two distinct organisms or to the pooled genomes of two distinct sets of organisms (see below).

Genomic profiling scans a complex biological sample for ID sequences, which are DNA fragments whose presence is indicative of a particular type of organism. Two types of ID sequences are useful for determining the presence of an organism. Group-specific sequences are common to essentially all organisms in a particular taxonomic group (i.e., within a biological group whose members are closely related by ancestry). In contrast, genomic difference sequences differentiate organisms in a particular taxonomic group. The useful diagnostic attribute of a family of genomic difference sequences is that unique subsets of the members of the family are present in the genomes of closely related strains in a group.

The diagnostic power of genomic profiling is, in part, due to its ability to test for a complex mixture of ID sequences that are characteristic of a large and diverse set of organismal types. It is therefore useful to expand on the earlier-presented definitions of such sets of diagnostic ID sequences.

A “family” of ID sequences is a set of group-specific and/or genomic difference sequences that is useful for identifying members of a particular group of organisms. The defining feature of the set of ID sequences in a family is that all of the members can hybridize to a single “distinct genome” (see Table 1 and definitions, above). For example, a family of ID sequences might consist of 100 ID sequences that include 80 genomic difference sequences that differentiate strains of the E. coli O157:H7 group of pathogens (but that are derived from a single strain, DEC3B), 18 group-specific sequences that are present in all E. coli O157:H7 strains, and 2 group-specific sequences that are present in all strains of the species E. coli. Note that, although the sequences are useful to uniquely identify pathogens in the E. coli O157:H7 group, all of these sequences can hybridize to one distinct genome: that of E. coli O157:H7 strain DEC3B.

A unique feature of genomic profiling is that it can be used to scan a sample for the presence of many different families at once. A set of ID sequences that is composed of more than one family is called an “ensemble” of ID sequences. The number of distinct groups of organisms tested for by an ensemble is reflected by the number of families in the ensemble. The number of families in an ensemble can, in turn, be accurately defined by a quantity called the “minimum genomic derivation” of an ensemble. The “minimum genomic derivation” is the minimum number of “distinct genomes” to which all of the sequences comprising the ensemble can hybridize. For example, genomic profiling can use an ensemble with a minimum genomic derivation of 5 to simultaneously test a sputum sample for the presence of Mycobacterium tuberculosis, Legionella spp, Coccidoides immitus, influenza virus, and respiratory syncytial virus. Thus, the ability of genomic profiling to identify a broad spectrum of organisms in a single test is a consequence of its ability to scan a sample for the presence of ID sequences in an ensemble that has a large “minimum genomic derivation.”

Similarly, in non-infectious disease applications, such as human genetic screening and forensics, genomic profiling can be used to scan a sample for an ensemble of single-nucleotide polymorphisms. An ensemble of SNPs is defined as a set of multiple families of SNPs analogously to the definition of an ensemble of ID sequences. A family of SNPs, like a family of ID sequences, reflects the genotype of a single individual. Note that, whereas a family of ID sequences is defined by the ability of the member ID sequences to hybridize to the genome of a single individual, a family of SNPs is defined by correspondence to the genotype of a single organism.

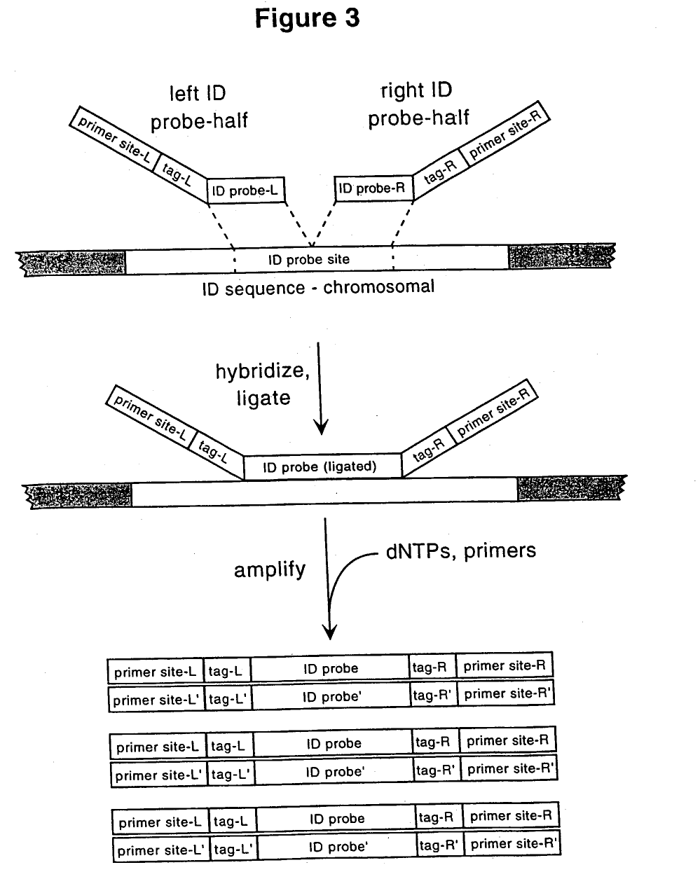

An advantage of genomic profiling as applied to genotyping is that SNPs can be detected using a robust hybridization assay. In some large-scale SNP genotyping applications, SNP genotypes are detected that discriminate between oligonucleotide hybrids which form perfect duplexes and those that form duplexes with a single base-pair mismatch. In contrast, the genomic profiling assay can test for the presence or absence of oligonucleotide tag sequences, a much easier task. To achieve this more robust hybridization assay, a unique non-biological tag sequence can be incorporated into each SNP probe. Thus, an ensemble of such SNP probes is congruent to an ensemble of tag sequences and each family of SNPs is congruent to a family of tag sequences. In the genomic profiling assay detection step, a detection ensemble, composed of an ensemble of tag sequences, can be used to detect a family of amplified SNP probes (comprising a congruent family of tag sequences) that corresponds to the genotype of a genomic DNA sample isolated from a single individual (see FIG. 3).

A Preferred General Configuration of the Genomic Profiling Method Consists of the Following Steps:

Step 1: Specifying an ensemble of ID sequences, comprising genomic difference sequences and group-specific sequences, that will be probed for in a given test. This step involves choosing the organisms to be detected and choosing families of diagnostic ID sequences.

Step 2: Designing and preparing an ensemble of probes corresponding to the ensemble of ID sequences to be detected in a biological sample. Control probes are also designed and prepared.

Step 3: Designing and preparing a detection ensemble corresponding to the ensemble of ID probes. Control sequences corresponding to the control probes are also designed and prepared. A two-dimensional detection array is prepared in one preferred embodiment.

Step 4: Preparing the biological sample. This step involves lysis of organisms in a sample so that nucleic acid molecules of the organisms become available for hybridization. For example, a sample, such as a stool or respiratory sample, is treated so that nucleic acid molecules from organisms in the sample are bound to a solid support.

Step 5: Selecting ID probes from the ID probe ensemble that hybridize (bind) to genomic sequences in the prepared sample. Non-hybridizing, unbound probes, are then removed by washing.

Step 6: Amplifying the ID probes that bind to the genomic sequences in the sample.

Step 7: Identifying the sample-selected ID probes by hybridization of the amplified probe sequences to a detection ensemble.

Step 8: Quantifying the target organisms in the biological sample by in situ of the sample-selected ID probes to the biological sample.

(Note that, for simplicity, the steps of the preferred general configuration are described with reference to genomic profiling using ID sequences. For the modifications of this procedure that are used for genomic profiling using SNPs, see example 5)

Each of These Steps is Described in Further Detail, as Follows.

Step 1: Specifying an ensemble of ID sequences, comprising genomic difference sequences and group-specific sequences, that will be probed for in a given test. This step involves choosing the organisms to be detected and choosing families of diagnostic ID sequences.

The first step in genomic profiling involves selection of the types of organisms to be detected. For example, for medical uses, one selects human pathogens; to test for food spoilage, one selects bacteria that cause food toxicity; for forensic purposes, one selects a variety of human individuals, and so on. The organisms chosen for a particular test can be widely different in their genetic makeup, such as members of different kingdoms (i.e., viruses, bacteria, archaebacteria, fungi, protozoa, plants, and animals); alternatively, the chosen organisms may be members of a smaller group, such as a species. A significant use of genomic profiling is in the identification of pathogens in a human bodily fluid sample, such as blood, urine, cerebrospinal fluid, or sputum, or in feces. (The method is also important as applied to numerous other tissue samples.) Depending on the source of tissue sample and the symptoms of the patient, a decision is made as to the important types of organisms to be identified. For example, one can choose to detect viruses, bacteria, and eukaryotic parasites that are common causes of pneumonia.

Once the types of organisms to be identified by the genomic profiling assay are determined, an ensemble of ID sequences are chosen for the assay. The ensemble is assembled from families of ID sequences, each of which is diagnostic of one type of organism to be detected in the assay. The ensemble of ID sequences need not necessarily be physically isolated. Rather, such an ensemble may be merely conceptualized to facilitate the design of ID probes for use in constructing a probe ensemble (see below).

As described above, the ensemble of ID sequences comprises two useful types of sequences: genomic difference sequences and group-specific sequences. For any particular type of target organism, the choice as to whether to include group-specific sequences, genomic difference sequences, or both depends on the diagnostic issues associated with the particular type of organism.

Group-specific sequences are most useful diagnostically when it is important to know if any member of a biological group is present in a sample. For example, group-specific sequences are helpful if it is important to know if any member of the group Salmonella enterica is present in a gastrointestinal sample. Group-specific sequences are also likely to be chosen when testing for a virus, such as Hepatitis C virus.

In contrast to group-specific sequences, genomic difference sequences are particularly useful when differentiation between closely related strains within a group is required. This is the case, for example, when an important pathogen (e.g., E. coli O157:H7) is closely related to strains (e.g., commensal E. coli) that occur in the same tissue as the pathogen. Genomic difference sequences are also valuable when a fingerprint of an infectious agent is desired. Fingerprinting, or high-resolution strain identification, can be a powerful epidemiological tool for tracking and containing infectious disease outbreaks, including hospital-based infections. Therapeutically, fingerprinting, especially in a rapid, culture-independent test, offers the potentially life-saving opportunity to determine which antibiotic to administer much faster than is done in current practice.

For each type of organism to be detected in a genomic profiling assay, a family of ID sequences comprising group-specific sequences and/or genomic difference sequences is selected using standard methods, such as those described below and in the examples. If the sequence of a newly isolated ID sequence is not already known, the sequence is determined by standard methods. Various families of ID sequences, corresponding to different, and possibly unrelated, types of organisms, are then organized into an ensemble.

An ensemble of probes corresponding to the selected ID sequences is then designed and synthesized, using commercially available oligonucleotide synthesis methods or services, by synthesis of recombinant DNA from plasmids, or by any other method for generating sufficiently pure DNA molecules. A probe for a given ID sequence can consist of one, two, or several oligonucleotides, as well as attached moieties for use in detection. At least part of the probe, the ID site, is designed to hybridize to ID sequence nucleic acid molecules from test organisms.

Isolating genomic difference sequences using genomic subtraction. Genomic difference sequences are used to distinguish one strain from a closely related strain. A family of genomic difference sequences has the property that different subsets of the sequences in the family are present in different strains. Genomic profiling can ascertain the subset of a family of genomic difference sequences that occurs in a clinical sample. In this way, a strain that is present in a sample is precisely identified. An advantage of the genomic profiling assay over the prior assays is that many different families, each capable of fingerprinting a particular group of organisms, can be surveyed simultaneously.

Genomic difference sequences that are useful for clinical diagnosis can be isolated by performing genomic subtraction on a pathogenic strain and a related, non-pathogenic strain. Some genomic difference sequences are of great clinical significance. For example, in recent years it has become clear that pathogenic bacteria frequently harbor “pathogenicity islands,” which are continuous stretches of DNA containing multiple virulence genes required for pathogenicity. Closely related non-pathogenic strains generally lack pathogenicity islands. Thus, pathogenicity islands are useful genomic difference sequences. Other, and perhaps most, genomic difference sequences have no clinical significance, but are nonetheless extremely valuable for strain identification. It is worth noting that the distinction between group-specific sequences and genomic difference sequences can sometimes be unclear. For example, an E. coli O157:H7 pathogenicity island sequence could be seen as a genomic difference sequence, as it occurs in some strains of E. coli, but not in others. Or, the same sequence could be viewed as a group-specific sequence, since it occurs in all members of the taxon composed of E. coli:O157:H7 strains. Regardless of the occasional ambiguity, these sequences are useful diagnostic ID sequences.

A family of genomic difference sequences can be isolated by using one of several genomic subtraction methods (e.g., Straus, 1995, supra; Diatchenko et al., Proc. Natl. Acad. Sci. U.S.A. 93:6025-6030, 1996; Tinsley et al., Proc. Natl. Acad. Sci. U.S.A. 93:11109-11114, 1996). Genomic subtraction isolates DNA sequences that occur in the genome of one strain (the “+” strain), but not in the genome of a related strain (the “−” strain). The products of genomic subtraction are a family of genomic difference sequences: the entire set hybridizes to the “+” strain, none hybridize to the “−” strain, and unique subsets hybridize to closely related strains. A general property a family of genomic difference sequences is that the members are found in different combinations in the genomes of strains that are closely related to the strains used to make the genomic difference samples (i.e., the strains used for the genomic subtraction). The unique subset of the family of genomic difference sequences that is present in an individual strain constitutes a high resolution fingerprint. Note, however, that the entire family of genomic difference sequences derived from a genomic subtraction can hybridize to a single strain, the one used to make the “+” genomic subtraction sample. (In cases in which more one strain is used to make the “+” genomic difference sample, the products of subtraction can constitute more than one family.)

Genomic subtraction generally employs subtractive hybridization and affinity chromatography to purify genomic difference sequences from the “+” and “−” genomic difference samples (Straus, 1995, supra). Genomic DNA from two related strains (the “+” strain and the “−” strain) is first prepared. The DNA from the “+” strain is cut with a restriction enzyme, and the DNA from the “−” strain is sheared randomly and modified with biotin, which is an affinity label that permits subsequent removal of the “−” strain DNA by binding to its ligand, avidin. Enrichment for genomic difference sequences is achieved by allowing denatured DNA fragments from the “+” strain and the “−” strain to reassociate. After reassociation, the biotinylated sequences—and all of the sequences that have hybridized to the biotinylated sequences—are removed by binding to avidin-coated beads. This subtraction process is then repeated several times. In each cycle, unbound DNA from the “+” strain from the previous round of subtraction is hybridized with fresh, biotinylated DNA from the “−” strain. The unbound DNA from the “+” strain from the final cycle is ligated to adaptors and is amplified by using one strand of the adaptor as a primer in the polymerase chain reaction. The amplified sequences can then be cloned. Note that performing the reciprocal subtraction (i.e., switching the “+” and “−” strains) produces a different set of genomic difference sequences. Such subtraction methods, which can be used to generate genomic difference sequences, are known to those skilled in the art of recombinant DNA technology, and such methods have been widely published. Additional details are provided in the Examples, below.

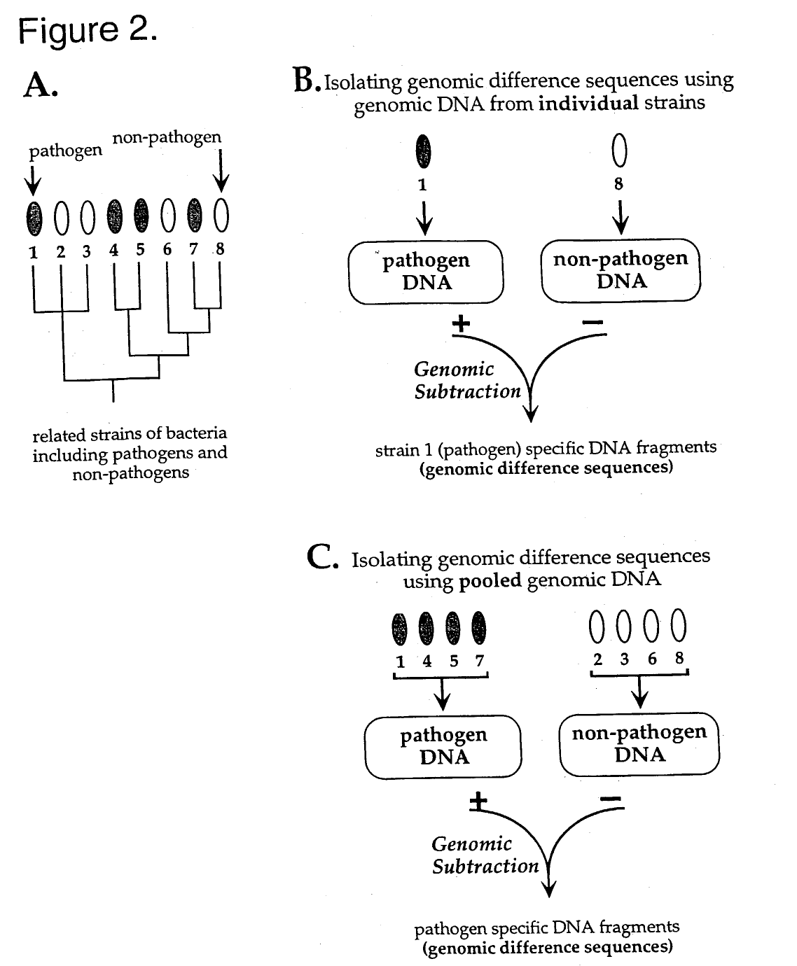

An overview of genomic subtraction is illustrated in FIG. 2. FIG. 2A shows a hypothetical phylogenetic tree of a group of organisms that share a common ancestry (a “taxon”). Some of the organisms are pathogens and some are non-pathogens. FIG. 2B illustrates one strategy for isolating genomic difference sequences. Two organisms in a group of related strains (e.g., strains 1 and 8) can be chosen to make the genomic difference samples. Strain 1, a pathogen, is used to make the “+” genomic difference sample and strain 8, a non-pathogen, is used to make the “−” genomic difference sample. The products of the subtraction (FIG. 2B) are genomic difference sequences that occur in strain 1, but not in strain 8. These genomic difference sequences are useful for fingerprinting any strain within the group (i.e., including strains 2-7). Genomic subtraction using strain 1 and strain 8 (FIG. 2A) may yield hundreds of sequences from strain 1 that are not present in strain 8. Strain 2 has some of these genomic difference sequences, but lacks others. Strain 5 harbors a distinct subset of the genomic difference sequences, as would strain 7, and so on. The important and general finding is that when genomic subtraction is applied to two strains in a group (strains 1 and 8 in FIG. 2 and the example described here), related strains (e.g., strains 2 and 5) harbor distinct subsets of the resulting genomic subtraction products.

As is illustrated in FIG. 2C, genomic difference sequences can also be generated by pooling genomic nucleic acid molecules from several organisms. For example, a “+” sample can be generated by pooling several pathogens, and a “−” sample can be generated by pooling several non-pathogens (FIG. 2C). In this case, the genomic difference sequences that are isolated by genomic subtraction are sequences that occur in at least one of the pathogen genomes of the “+” genomic difference sample but none of the non-pathogen genomes of the “−” genomic difference sample.

Instead of using subtractive hybridization, a computer and sequence comparison software can be used to compare the genomes of two organisms or two sets of organisms, and thus to generate genomic difference sequences. This method is practical, for example, when the sequence of the genome of the target organism is complete or is essentially complete. For example, a computer-based comparison of related strains of Helicobacter pylori, whose sequences have recently been completed, has been reported (Alm et al., Nature 397:176-180, 1999). The published analysis and publicly available data provide numerous genomic difference sequences that are unique to one or the other strain. This analysis, then, constitutes a type of “virtual” genomic subtraction analysis from which genomic difference sequences have been determined.

Isolating group-specific sequences. When it is important to determine only whether any member of a certain group is in a biological sample (as opposed to determining which individual strain from within a certain group), group-specific sequences are included in the ensemble of ID sequences that is assessed by the genomic profiling assay. Group-specific sequences can be isolated in numerous ways, including by genomic subtraction and by analysis of public databases. For example, a genomic subtraction using DNA from a pathogenic Mycobacterium tuberculosis strain as the “+” genomic difference sample and the DNA from a non-pathogenic Mycobacterium strain as the “−” strain yields group-specific sequences that include virulence genes that are common to all pathogenic Mycobacterium tuberculosis strains. These group-specific sequences are valuable ID sequences for testing for the presence of strains that cause tuberculosis. As another example, group-specific sequences for herpes simplex virus can be isolated by scanning the viral genomic DNA sequences in a public database, such as GenBank, for sequences that occur in all known isolates of herpes simplex virus, but in no other type of virus in the database.

Step 2: Designing and preparing an ensemble of ID probes corresponding to the ensemble of ID sequences to be detected in a biological sample. Control probes are also designed and prepared.

In the second step of genomic profiling, an ensemble of ID probes is designed such that ID probes in the ensemble can hybridize to members of the ensemble of ID sequences that are chosen for the genomic profiling assay in Step 1. An ID probe can consist of a single oligonucleotide or, in a preferred embodiment, two or more oligonucleotides. An ID probe and any of its constituent oligonucleotides can comprise one or more functional portions.

A portion of an ID probe, the ID site, corresponds to an ID sequence. In a preferred embodiment of the method, the ensemble of ID probes contains multi-functional ID probes in which the first portion of a probe sequence corresponds to one sequence in the ID sequence ensemble that is assembled in Step 1. Thus, one such ID probe includes a sequence or a set of sequences that corresponds to a portion of an ID sequence, and can hybridize to nucleic acid molecules including the ID sequence, as is described below. This portion is called an ID site. For example, such an ID probe can contain an ID site that correspond to a genomic difference sequence or a group-specific sequence.

A portion of an ID probe corresponds to amplification sequences. An important advantage of genomic profiling is its ability to achieve robust artifact-free amplification of many sequences at once. The genomic profiling assay avoids the usual amplification artifacts that arise during multiplex amplification by using a very small number of amplification sequences to direct the amplification of a large number of distinct ID probes. To this end, a second portion of the ID probe (in addition to the first portion, which corresponds to an ID sequence) can include one or more amplification sequences. This second portion can, for example, correspond to one or more primer binding sites, or to a binding site for a nucleic acid polymerase, such as Qβ replicase. The amplification moieties are common to most or all of the probes in the ensemble (including control sequences) that are to be amplified. Therefore, the set of probes including the ensemble of ID probes and the control sequences (see below) can be efficiently amplified in the same reaction.

A third, optional, portion of the probe can include a tag sequence that is used in detection of the amplified probe. The use of tags is discussed under Step 3, below.

Control sequences. Both positive and negative controls can be included with an ensemble of ID probes. There can be positive control sequences included with the ensemble that do not correspond to sequences in actual genomes, but rather that correspond to control nucleic acid molecules that are added to the sample during sample preparation. Detection of the positive control sequences in the genomic profiling assay indicates that the entire assay is working correctly. (When there are no ID sequences detected in a sample, it is important to know if there are truly no ID sequences present in the sample, or alternatively if the assay failed for some reason.)

Negative control sequences can also be included with the ensemble of ID sequences probes. These negative control sequences do not correspond to naturally occurring sequences and, in contrast to positive control sequences, are not added to the biological sample. The level of negative control sequences detected by the genomic profiling assay indicates the level of background in the assay due to ID sequence-independent selection and amplification of ID probes.

Binary probes (probe-halves). In one embodiment, an ID probe consists of a pair of oligonucleotides, the left and right ID probe-halves (FIG. 3). The inner portion of each right and left probe-half includes a sequence that corresponds to adjacent parts of an ID sequence, such as a genomic difference sequence or a group-specific sequence. When the probe-halves hybridize to the denatured ID sequence, the probe moieties can be joined by a nucleic acid ligase. As is described below, the sample-dependent ligation of probe-halves results in the formation a larger molecule that can be amplified and detected.

In this embodiment, the outer portion of each probe-half comprises an amplification sequence, for example, a site corresponding to a primer binding site for the polymerase chain reaction. In an ensemble of such ID probes, each probe has a unique ID and tag sequence, but a common pair of primer binding sites. If a tag sequence is present, it is located between the inner and outer portions in one of the probe-halves.

FIG. 3 illustrates the embodiment using probe-halves, ID sequence-dependent ligation, tags, and PCR amplification of probes-halves that hybridize to the sample. In this example, the left primer for PCR is identical to the primer site-L sequence, and the right primer is the reverse complement of the primer site-R sequence. Four different tag sequences (tag-R, tag-R′, tag-L, and tag-L′) can be included in the detection array (see below). The four tag sequences hybridize to the two complementary sequences comprising each of the two tag sequences in the amplified ID probes.

ID probe synthesis and concentration. ID probes are prepared by standard nucleic acid synthesis techniques. The sequences and concentrations of the ID probes in an aqueous solution are defined. The concentration of the ID probes in an aqueous solution can be varied according to need. For example, in an ensemble of ID probes, each oligonucleotide can be present in an equimolar amount. In an alternative embodiment, an ID probe is present in an amount that is inversely related to the expected abundance of its corresponding ID sequence in a typical biological sample that contains the corresponding organism. For example, if a person has a gastrointestinal infection with both rotavirus and parasitic nematodes, the copy number of rotavirus genomes in a stool sample is likely to be greater than the copy number of nematode genomes in the stool sample. It may therefore useful to have probes for rotavirus sequences present in limiting amounts.

Step 3: Designing and preparing a detection ensemble corresponding to the ensemble of ID probes. Control sequences corresponding to the control probes are also designed and prepared. A two-dimensional detection array is prepared in one preferred embodiment.

The role of the detection ensemble is to detect and identify the subset of the ensemble of ID probes that are selected by hybridization to ID sequences in the biological sample. The detection ensemble comprises sequences corresponding to the ensemble of ID probes assembled in Step 2 (and to the ID sequences that are diagnostic for the presence of various types of organisms in the test). In other words, the detection ensemble is congruent to the ensemble of ID probes. Control sequences corresponding to the control probes are also included with the detection ensemble.

The detection ensemble consists of nucleic acid molecules that can be used to detect probe-sample hybridization events. The detection ensemble can include sequences that correspond to ID sequences or to sequence tags within the probes. In one embodiment of the genomic profiling method, the detection ensemble DNA sequences are denatured and fixed to a solid support, so that the detection ensemble DNA sequences can hybridize with added ID probes. This detection ensemble, when constructed on a planar solid support, is termed a two-dimensional detection array. The detection sequence DNAs are placed in different positions on the support. Methods for fixing DNA molecules to solid supports in this manner are known to those of skill in the art of genomics. For example, the methods referred to in the Examples can be used for this purpose. Alternatively, hybridization of the sample-selected ID probes to the detection array may be carried out in the liquid phase, as is described in Example 3 below.

In a preferred embodiment of array design, detection sequences that correspond to a group or related groups are positioned near each other on the array. Thus, families of detection sequences, i.e., those that are specific for a given type of organism (for example, pathogens in the group E. coli O157:H7) are deposited as a group of neighboring spots. Furthermore, families of detection sequences corresponding to closely related families (for example, E coli O157:H7 and Shigella) are positioned in the same region of the array. This organization facilitates readout of the hybridization results.

Positive and negative control sequences that are included with the ID probe ensemble (see above) may also be incorporated into the detection ensemble. As discussed above, the positive control sequences are also mixed with the biological sample and are used to indicate the proper functioning of the assay. The positive control probe sequences hybridize to the target control sequences in the biological sample, are amplified, and then hybridize to the corresponding control sequences in the detection array.

The negative control sequences are a useful measure of the pathogen-independent background signal in the assay (i.e., a measure of the amount of ID probe that is amplified in spite of the absence of the corresponding pathogen in the biological sample). Negative control sequences, in contrast to positive control sequences, are not mixed with the biological sample. Thus, negative control probe sequences have no target sequence to hybridize to in the biological sample. Non-specific association of the negative control sequences with the biological sample or the sample matrix permits subsequent amplification and hybridization of these sequences to the corresponding sequences in the detection array.

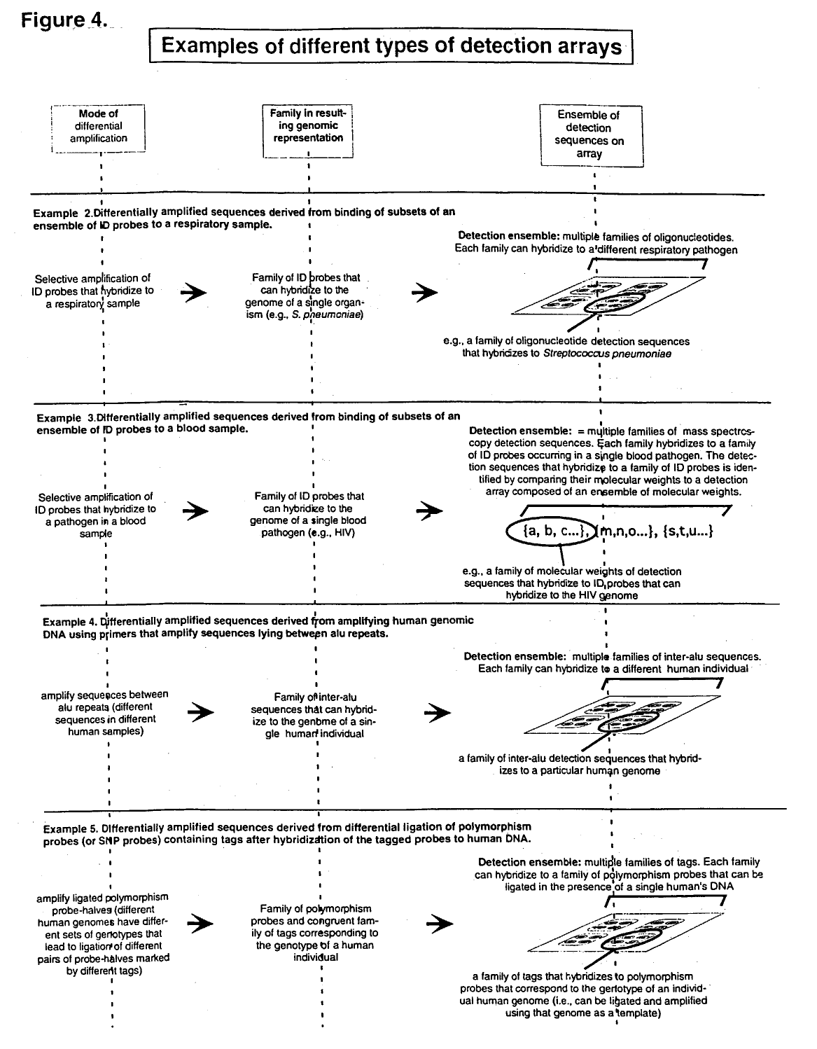

Fabrication of an array containing an ensemble of detection sequences. Various types of detection arrays can be used to detect diagnostic sequences. FIG. 4 illustrates some designs of detection arrays that are used in the examples described below.

Numerous methods for constructing arrays of nucleic acid molecules have been described. A preferred method for use in the present invention is one in which nucleic acid molecules are deposited at a high-density on polylysine treated glass slides (see, e.g., Schena et al., Science 270:467-470, 1995). Detection sequences corresponding to ID sequences can be deposited in the arrays as cloned DNA (e.g., as inserts in a plasmid vector), as amplified DNA (e.g., the PCR products resulting from amplification of cloned sequences), or as synthetic oligonucleotides.

Alternatively, the detection ensemble can include an addressable set of synthetic oligonucleotide tags, rather than ID sequences. The tags, in this case, correspond to tag elements in the ID probes (as is described below) or SNP probes (as described in example 5). Each addressable tag in the array corresponds to the tag joined to a specific probe sequence in the ensemble of probes subjected to hybridization-selection (see below). The one-to-one relationship between array elements and the probe ensemble makes it possible to identify the ID sequences in a mixture by observing which oligonucleotide tag array elements hybridize to molecules in the mixture. Advantages of this approach are that prefabricated arrays can be used, as arrays containing the same set of addressable tags can be used for different sets of probes. For example, a set of probes for detecting respiratory pathogens and a set of probes for detecting gastrointestinal pathogens can use the same set of tags. Thus, a single array can be used for identifying pathogens in respiratory or gastrointestinal tract samples.

Alternatively, the detection array can be a set of detection sequences that are hybridized in liquid to the sample or probes. Detection arrays can also be a set of physical properties, such as molecular weights, to which diagnostic products are compared.

Step 4: Preparing the biological sample. This step involves lysis of organisms in a sample so that nucleic acid molecules of the organisms become available for hybridization. For example, a sample, such as a stool or respiratory sample, is treated so that nucleic acid molecules from organisms in the sample are bound to a solid support.

The aims achieved by the following sample preparation strategy are:

(a) Converting samples from a broad range of sources (e.g., culture, colonies, sputum, blood, urine, and feces) into a common form that is compatible with subsequent steps of the assay. Organisms are lysed and their genomic nucleic acid molecules are made available for hybridization.

(b) Concentrating the sample, thereby increasing the sensitivity of the assay when testing for organisms in dilute form (e.g., in the case of urine or blood samples).

(c) (c) Eliminating or attenuating the effects of enzymatic inhibitors in the sample by removing or immobilizing inhibiting substances.

Any of several methods of sample preparation can be used to prepare the sample for use in the present methods. The general idea of sample preparation is to liberate and to denature nucleic acid molecules, and to remove contaminating proteins and other materials that can interfere with subsequent steps. Sample preparation methods can, optionally, be used to selectively retain DNA, RNA, or both.

Before preparation, dilute sample types, such as urine samples, can be concentrated by filtration through standard filtration units. If the sample source contains particulate matter that is greater than the organisms of interest, the particles are removed from the sample before the sample concentration step is carried out, by filtering the sample through a filter with pore sizes larger than the organisms of interest. When testing for microorganisms, for example, pre-filtering through a membrane with an average pore size of 20 to 30 microns is used to separate large particles from microorganisms.

Alternatively, centrifugation steps can be used to separate microorganisms from material having different size or density. For example, large particulate matter can be separated from microorganisms by a centrifugation step at a speed that causes large particles, but not microorganisms, to be deposited in a pellet. Microorganisms are, optionally, separated from the liquid phase by centrifugation, e.g., in the case of cultured microbiological samples. A combination of filtration and centrifugation is used to concentrate and enrich for suspected test organisms. Pellets recovered from samples processed by centrifugation are then prepared further. Both filtration and centrifugation have the potential disadvantage that viruses can be lost from samples. Other enrichment methods such as affinity chromatography, cell-sorting, and antigen-based enrichment may also be included this step.

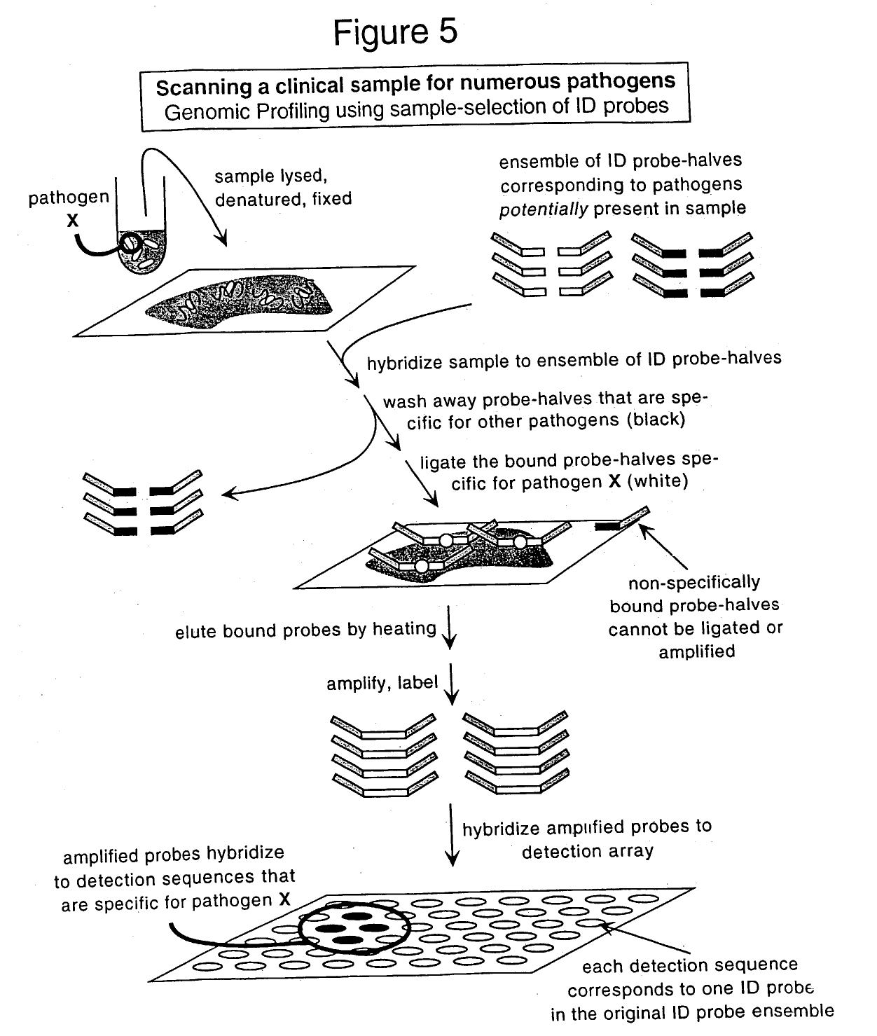

In a preferred embodiment, experimental samples (obtained by filtration or centrifugation, as well as crude samples with a high content of microbes, such as fecal samples) are deposited and fixed to a solid support, such as a nylon filter, particulate matrix, or beads (FIG. 5). Use of a solid support provides several advantages over other methods. The sample DNA is fixed to a solid support and denatured in preparation for hybridization to single stranded nucleic acid molecule probes. By immobilizing and washing crude DNA samples, inhibitors of enzymatic steps (e.g., ligation and amplification) are either immobilized on the matrix or washed off of the filter containing the bound DNA. This is an important advantage, as PCR tests on clinical samples sometimes lack sensitivity, due to inhibition by sample components. Finally, it is simple to include internal controls for detecting false negative results.

The preferred support is a nylon filter, which is durable but flexible, and is extensively used for fixing nucleic acid molecule-containing samples for hybridization assays (Church et al., Proc. Natl. Acad. Sci. USA 81:1991-1995, 1984). Crude samples, such as sputum or fecal samples, are smeared onto a solid support, as is currently the practice when testing sputum samples for M. tuberculosa using the “acid fast smear” assay (Koneman et al., Color Atlas and Textbook of Diagnostic Microbiology (Lippincott-Raven, Philadelphia, 1997)). Similarly, colonies of bacteria or fungi growing on semisolid media on a petri dish can be “lifted” onto a nylon filter or smeared onto a filter from a petri dish smeared on a solid support.

In a preferred embodiment, samples are next fixed to the solid support using procedures that break open cells and denature any double stranded DNA in the sample. Numerous methods for breaking open cells have been developed. These include mechanical disruption and treatment with base, chaotropic agents, heat, and organic solvents. This step of the invention may incorporate one or more such methods for disrupting cells. A simple method, involving alkali treatment, followed by neutralization and washing, is a preferred means for fixing denatured DNA in a sample to a solid support (Hanahan et al., Methods Enzymol. 100:333-42, 1983; Grunstein et al., Proc. Natl. Acad. Sci. USA 72:3961-3965. 1975; Ausubel, 1987, supra).

If an assay yields a negative result, it is important to know whether the sample is truly free of genomic DNA from test organisms or whether the assay itself failed, i.e., whether the result is a false negative. False negatives can occur due to the presence of inhibitors in the experimental sample that block one of the enzymatic steps in the assay.

To identify false negative results, one or more positive control DNA samples can be added to the experimental sample. The positive control DNA samples contain DNA sequences that do not occur in the range of organisms being tested. Probes corresponding to the positive control DNA samples are included in the probe ensemble. These probes will be amplified and detected in all assays, unless one or more of the assay steps is unsuccessful. Failure to detect a signal from a positive control thus can indicate a false negative result.

FIG. 5 illustrates sample preparation, hybridization-selection, amplification, and detection of selected probes. In this embodiment, a sample is prepared by lysis onto a nylon filter so that the nucleic acid molecules of the sample are denatured and attached to the filter. A positive control DNA sample is also bound to the filter. Ligatable probe-halves are then hybridized to the bound nucleic acid molecules. If both halves of a probe bind to an ID sequence, they are ligated together to create a full-length probe, which can be PCR-amplified because there are primer binding sites at each end of the full-length probe. Incorrectly bound probe-halves cannot be amplified by PCR.

Step 5: Selecting ID probes from the ID probe ensemble that hybridize (bind) to genomic sequences in the prepared sample. Non-hybridizing, unbound probes are then removed by washing.

The goal of hybridizing the probe ensemble to a fixed sample is to select probes that correspond to, and thus can be used to identify, genomic DNA in the fixed sample, and to separate these hybridizing probes from the non-hybridizing probes. The genomic DNA of various target organisms hybridizes to distinct subsets of the ID probes. Thus, the particular subset of ID probes selected constitutes a fingerprint of the genome of a particular organism. The ID probe hybridization step is designed to be rapid, to be specific, and to test for a broad range of organisms. Inclusion of positive and negative controls facilitates determination of whether the hybridization is working as desired.

In this step, an ensemble of ID probes is hybridized to the denatured nucleic acid sample. Hybridization can be done in aqueous solution or with nucleic acid molecules that are immobilized onto a solid support, as is described above. Hybridization is performed by mixing the probe ensemble with the prepared biological sample, and preferably incubated until at least one Cₒt_{1/2} time period has elapsed. The probe/sample mixture is then washed, diluted, or otherwise treated so that unhybridized and non-specifically hybridized probe molecules are separated from the hybridized probe and the sample. Hybridized probes can be subjected to enzymatic treatment, such as ligation or nucleic acid polymerization. Finally, hybridized probes are separated from sample nucleic acid molecules and amplified, as is described in the next step.

In a preferred embodiment, a sample, including positive control nucleic acid molecules, is fixed on a solid support (FIG. 5). The sample is hybridized with an ensemble of probes, including ID probes, and positive and negative controls. The probes consist of pairs of oligonucleotides that hybridize to adjacent portions of an ID sequence. The hybridized sample is washed to remove unbound probes, and then is treated with a nucleic acid molecule ligase to ligate the left and right half-probes. Finally, the ligated left and right half-probes are removed from the sample and subjected to amplification. The following is a description of a particular version of this preferred embodiment.

i. Place the ID probe hybridization mixture over the experimental sample, which is affixed to a solid support, such as a glass slide or nylon filter. The preferred hybridization mixture includes:

a) An ensemble of ID probes, including genomic difference sequence and/or group-specific sequence probes. In this case, the ID probes are pairs of oligonucleotides consisting of two ligatable probe-halves. The preferred concentration of each of the half-probes is 1-10 nM, in a preferred volume of 10-100 μl. This probe concentration, under the preferred reassociation conditions, leads to an acceptable level of hybridization to the fixed sample within several minutes (Britten, et al., Meth. Enzym. XXIX: 363-418, 1972).

b) One or more pairs of positive control probe-halves at a concentration comparable to that of the ID probes. The sequences of these probes correspond to the positive control DNA fixed to the solid support (to which the biological sample is also bound).

c) One or more pairs of negative control probe-halves at a concentration comparable to that of the ID probes. These probe sequences have no counterparts in the fixed DNA sample.

d) 1 M NaCl/10 mM EPPS/1 mM EDTA, pH 8.0. Substitution of standard hybridization solutions is also acceptable (Ausubel, 1987, supra; Church, 1984 supra).

ii. Cover the hybridization mixture with a glass coverslip, preferably separated from the sample by a gasket (e.g., Cenegator™, catalog #009917, BioWorld Fine Research Chemicals).

iii. Incubate at approximately 65° C. for 5-30 minutes.

iv. Wash off the unbound probe. This is accomplished by removing the coverslip and washing the fixed sample under stringent conditions, such that only ID probes that reassociate with no, or few, mismatches remain bound to the fixed, complementary genomic DNA. The conditions chosen depend on several factors, including the length of the ID sequences in the probes and the degree of mismatch deemed acceptable.

v. Ligate the annealed pairs of probe-halves. T4 DNA ligase (e.g., from New England Biolabs) is used to ligate adjacent probe-halves that have annealed to complementary genomic DNA in the fixed experimental sample. The ligation is carried out according to the manufacturer's specifications.

vi. Remove the ligated probe-halves from the experimental sample. Probes that have annealed to complementary genomic sequences in the fixed experimental sample are eluted from the sample by brief incubation under denaturing conditions. Applying 10 mM EPPS/1 mM EDTA, covering with a coverslip, and heating briefly to 100° C. is a preferred method for releasing the bound probes.

Step 6: Amplifying the ID probes that bind to the genomic sequences in the sample.

The amplification step is the basis of the high sensitivity of the genomic profiling assay. (However, amplification may not be required in all applications.) After removing (by thermal or chemical denaturing) any ID probes that have hybridized to the biological sample, the ID probes are amplified using a nucleic acid polymerase and nucleic acid molecule precursors. Amplification can be primer driven, employing primer binding sites present in the probes. Alternatively, amplification can be driven by binding of specific nucleic acid polymerases, such as Qβ replicase or T7 RNA polymerase, to specific binding sites incorporated into the probes. Any of several amplification methods can be used, including the ligase chain reaction, PCR, ligation-dependent PCR, transcription-mediated amplification, strand-displacement amplification, self-sustaining sequence replication, rolling-circle amplification, etc.

The amplified products can be labeled during amplification. For example, the amplified products can be labeled either by using primers synthesized with a chemical label-(e.g., biotin or alkaline phosphatase) or a fluorescent label, or by using a labeled dNTP precursor. One particularly useful method is to use primers synthesized with a biotin end-label.

In a preferred embodiment of the method that includes ligation (FIGS. 3 and 5), there are a left primer and a right primer, which correspond to outer portions of the probe oligonucleotides. The left primer is identical to the outer portion of the left probe-half, while the right primer is the reverse complement of the outer portion of the right probe-half. Unligated probe-halves in the reaction mixture are not amplified to a significant extent. (The unligated left-halves of the probe-pairs have no complementary primer and are not amplified; the unligated right-halves of the probe-pairs are amplified linearly.)

Step 7, Identifying the sample-selected ID probes: hybridization of the amplified probe sequences to a detection ensemble.

To generate a fingerprint that is representative of the genome(s) present in the experimental sample, the sample-selected amplified ID probes must be identified. The identities of the selected ID probes are deduced by hybridization to an ensemble consisting of ID sequences or ID oligonucleotides or tags that correspond to (are congruent) the ID probes in the original unselected probe mixture. The sequences in the ensemble can correspond to portions of ID sequences or to tag sequences that are incorporated between the inner and outer portions of a probe. Design and construction of a detection ensemble is described in Step 3, above.

Identification of the amplified ID probes can be carried out using any of a variety of procedures. In one embodiment, the amplified ID probes are used to select members of a detection ensemble by hybridization in liquid medium. The selected detection ensemble members are then identified by determining their molecular weights using mass spectroscopy. The selected sequences are then identified by comparison to the list of molecular weights of the full ensemble of detection sequences. In a preferred embodiment, labeled amplified ID probes are identified by hybridization to a two-dimensional detection array (see Step 3 above). Standard procedures are used for hybridizing and detecting nucleic acid molecules (Ausubel et al., 1987, supra). Procedures for identifying the amplified ID probes are further described in the Examples below.

Step 8. Quantifying the target organisms in the biological sample by in situ hybridization of the sample-selected ID probes to the biological sample.

Quantifying the number of target organisms in a biological sample is often important. In medicine, for example, knowledge of human immunodeficiency virus concentration in the blood (also referred to as the viral load, or titer) is important for gauging the stage of the disease and the response to therapy. Knowledge of the numbers of target organisms in a sample can also be important when distinguishing between chance contamination of a sample and a bona fide infection.

The labeled ID probes that are used in Step 7 can be used to quantify the target organisms in the biological sample by using in situ hybridization methodology. A portion of the labeled, amplified, sample-selected ID probe mixture is denatured and used to hybridize to the fixed (and optionally stained) biological sample. Alternatively, any group-specific sequence(s) that is specific for the type of organism detected by the steps above can be used as a probe. For in situ hybridization, it is preferred to use a sensitive method, e.g., one using catalyzed reporter deposition that is powerful enough to detect single cells/viruses using single copy sequences, yet one that is easy to implement (e.g., Huang et al., Modem Pathology 11:971-977, 1998). The fixed sample may be the same sample that was used in Step 4, or may be prepared by other standard methods known to those familiar with the art (e.g., Nuovo et al., supra).

These Methods are Described in the Following Examples:

### EXAMPLE 1

### Testing a Gastrointestinal Sample for the Presence of Pathogens

Gastroenteritis. Gastrointestinal illness is a major international health problem. About 1 billion cases occur each year in children, resulting in about 5 million deaths. Certain forms of the illness can be fatal within several hours of the onset of symptoms. A diverse array of pathogens cause gastrointestinal illness, including bacteria, viruses, and protozoa. Rapid and accurate identification of pathogens that cause gastrointestinal illness is important for choosing an appropriate antimicrobial therapy, identification of hospital-acquired infections, and tracking outbreaks of food-borne pathogens, such as the newly emerged pathogen E. coli O157:H7.

Current methods for diagnosing gastrointestinal illness are far from ideal. Determining the identity of the infectious agent is often difficult, time consuming (usually requiring at least several days, and sometimes even weeks), and expensive, due to the number and range of possible pathogens (e.g., viral, bacterial, and parasitic pathogens). The presence of diverse microbes in the normal gut exacerbates the difficulty of identifying the cause of gastroenteritis. Testing for protozoan, viral, and bacterial infections, and examining samples for the presence of diagnostic human cells, requires different specialized laboratory facilities. Furthermore, highly trained personnel must be employed to carry out these tests.

Objectives and advantages. In this example, I use a single genomic profiling assay to test for the presence of a broad range of gastrointestinal pathogens in a sample from a patient with gastrointestinal illness. By simultaneously and rapidly (e.g., several hours) testing for common bacterial, viral, and protozoan pathogens, and for the presence of diagnostic human cells, the method offers a substantial improvement over current practices. The test helps in the determination of an appropriate and timely therapy. Furthermore, the genomic profiling assay is a powerful tool for epidemiological analysis, because it can produce high-resolution fingerprints.

Note that the genomic profiling assay described in this example to test clinical samples for gastrointestinal pathogens is also a valuable tool for the food testing industry. Testing for gastrointestinal pathogens in food is important for preventing gastrointestinal illness.

Overview of the example. A genomic profiling assay is developed that, in a single test, scans a gastrointestinal sample for the presence of a comprehensive set of gastrointestinal pathogens. I isolate an ensemble of ID sequences from various gastrointestinal pathogens. For bacterial pathogens and parasites, genomic subtraction is used to isolate genomic difference sequences and group-specific sequences. Group-specific sequences for identifying gastrointestinal viruses are isolated using computer analysis. The subset of the ensemble of ID sequences that are present in the DNA of a given pathogen constitutes its genomic profiling fingerprint. A fingerprint database is constructed by determining the subset of genomic difference sequences present in representative strains from each group of gastrointestinal pathogens. The identity of pathogens in a clinical sample is determined by comparing the genomic profiling fingerprint of the clinical sample to the database of fingerprints.

Overview of the methods used in the example. I use a variation of the genomic subtraction method of Straus et al. (Proc. Natl. Acad. Sci. USA 87:1889-1893, 1990) to identify pathogen-specific ID sequences from bacteria and parasites that cause gastrointestinal illness. Alternative methods can be used to isolate genomic difference sequences, and can thus be substituted for the subtraction technique outlined below. For viruses that cause gastrointestinal illness, I identify group-specific ID sequences using computerized search of sequence databases. The ID sequences in a particular sample are identified by hybridizing an ensemble of ID probes with the fixed genomic DNA of the sample. A subset of the ID probes will hybridize, and thus be retained by the fixed genomic DNA. The hybridized ID probes are amplified using a ligation-dependent PCR strategy. The identity of the amplified ID probes is determined by hybridizing them to a detection ensemble, which, in this case, is an ordered two-dimensional array of the entire, unselected set of ID sequences. The pattern of hybridization signals visualized on the array constitutes a genomic profiling fingerprint.

Isolating Genomic Difference Sequences from Bacteria that Cause Gastrointestinal Illness

Strategy for isolating ID sequences from bacteria. For diagnosing gastrointestinal illness, the most useful diagnostic ID sequences are those that are present in gut pathogens, but absent in the hundreds of species that populate the healthy intestine. For many bacterial gastrointestinal pathogens, such ID sequences can be effectively isolated using genomic subtraction. The genomic subtraction strategy used depends on the particular pathogen, as is discussed above (Step 2 in detailed description-section). This section illustrates two different strategies used to isolate genomic difference sequences for Salmonella enterica and E. coli, which are representative gastrointestinal pathogens.

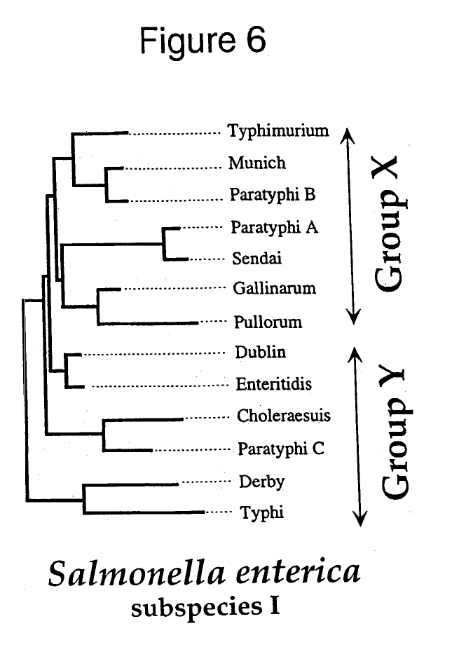

Strategy for isolating genomic difference sequences from Salmonella enterica. More than 99% of clinical isolates of the genus Salmonella are members of the subspecies Salmonella enterica. All strains of Salmonella enterica are considered to be human pathogens. Therefore, this group typifies those taxa (biologically related groups) for which identifying and distinguishing any member of the group from any other member is the diagnostic goal. There are many ways to use existing strains to isolate markers for high-resolution identification; this example uses the strategy illustrated in FIG. 6.

For this approach, the subspecies of Salmonella enterica are divided into two subgroups, Group X and Group Y. DNA from the representative members of each subgroup are pooled to construct a genomic difference sample for Group X and a genomic difference sample for Group Y. Strains from each branch are obtained from the SARB reference collection (Boyd et al., J. Gen. Microbiol. 139:1125-1132, 1993). Reciprocal subtractions using the genomic difference samples are executed. In one subtraction, the X genomic difference sample serves as the “+” sample and the Y genomic difference sample serves as the “−” sample. The products of this subtraction are sequences found in at least one member of group X, but not found in any member of group Y. In the reciprocal subtraction experiment, the Y genomic difference sample serves as the “+” sample and the X genomic difference sample serves as the “−” sample. The products of this subtraction are sequences found in at least one member of group Y, but not found in any member of group X.

The genomic difference sequences that are isolated by this genomic subtraction strategy constitute one or more families. In general, the strategy yields more than one family, i.e., all of the ID sequence subtraction products generally cannot hybridize to any single genome. Genomic subtraction of pooled organisms is thus an effective method to generate multiple families of ID sequences from within a group of related organisms.

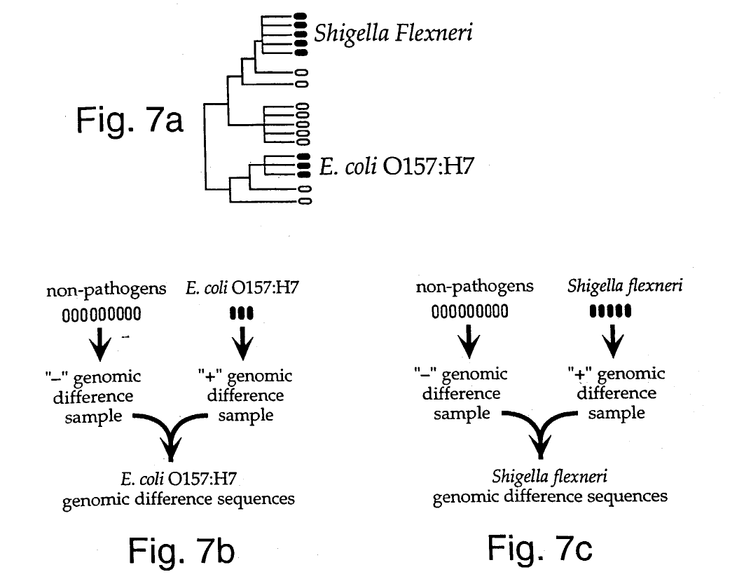

Strategy for isolating genomic difference sequences from E. coli. Part of the phylogenetic tree of the E. coli group is shown in FIG. 7A. Note that the pathogens (black) in this group (E. coli O157:H7 and Shigella flexneri) have very closely related sibling taxa that are not pathogenic (white). This is also the general case for the part of the E. coli phylogenetic tree that is not shown in the figure. The presence of numerous non-pathogenic or commensal E. coli in the gut of healthy individuals can confound the diagnosis of a pathogenic strain of E. coli: E. coli typifies groups of organisms that are found in humans and that contain both pathogens and non-pathogens.

To isolate genomic difference sequences for fingerprinting such groups, the strategy depicted in FIG. 7B and FIG. 7C is applied. Representative strains from the non-pathogenic taxa (branches) are pooled and their DNA is used to make the “−” genomic difference sample. Representative strains from the pathogenic taxa (branches) are pooled and their DNA is used to make the “+” genomic difference samples.

The products of genomic subtraction are sequences found in at least one member of the pathogen group (either E. coli or Shigella flexneri), but not found in any non-pathogenic strain in the subtraction. Note that this genomic subtraction will isolate genomic difference sequences, some of which are also group-specific sequences, in that they occur in all members of a group (e.g., E. coli O157:H7), but not in members of related groups. Virulence genes, i.e., those that are involved in the infectious process, that occur in the pathogenic E. coli (but not in non-pathogenic E. coli) fall into this class of products.

Strains for this experiment are from the ECOR (non-pathogenic) and DEC (pathogenic) strain collections provided by Dr. Thomas Whittman (Penn. State University).

TABLE 3
Pathogens that cause acute gastrointestinal illness.

Bacteria
Escherichia coli
Salmonella
Shigella
Yersinia enterocolitica
Vibrio cholera
Campylobacter fecalis
Clostridium difficile
Viruses
Rotavirus
Norwalk virus
Astrovirus
Adenovirus
Coronavirus
Parasites
Giardia lamblia
Entamoeba histolytica
Blastocystis hominis
Cryptosporidium
Microsporidium
Necator americanus
Ascaris lumbricoides
Trichuris trichiura
Enterobius vermicularis
Strongyloides stercoralis
Opsthorchis viverrini
Clonorchis sinensis
Hymenoplepis nana

Bacterial pathogens that cause gastrointestinal illness. Table 3 lists common groups of bacteria that cause gastrointestinal illness. Infections caused by some of these pathogens, including Vibrio cholera and enterohemorrhagic E. coli (e.g., E. coli O157:H7), can be fatal, even in healthy individuals. Rapid diagnosis is a key to effecting appropriate treatment and containing outbreaks.

To isolate families of ID sequences from the groups of bacteria listed in Table 3, I use the strategies applied to E. coli and Salmonella that are described above.

Preparing genomic DNA for subtractions. To prepare DNA to make the genomic subtraction samples, strains listed in Table 3 are grown to saturation in liquid culture (500 ml) and genomic DNA is prepared (Ausubel et al., 1987, supra). “+” and “−” strains are chosen by the same considerations described above for E. coli and Salmonella. DNA (50 μg) from each “+” strain is combined (henceforth, referred to as the “+” DNA). Similarly, DNA (50 μg) from the “−” genomic difference sample strains are combined (henceforth, referred to as the “−” DNA).

Preparing genomic difference samples. To make the “−” genomic subtraction samples, the DNA is sheared, reacted with photobiotin acetate, and resuspended at 2.5 mg/ml, as was described previously (Straus, 1995, supra). The “+” genomic subtraction samples are prepared by cutting “+” DNA (2 μg) with the restriction enzyme Sau3A, which generates fragments having sticky ends. After precipitating with ethanol, the DNA fragments are resuspended in 10 mM EPPS/1 mM EDTA, pH 8.0 (EE) at 0.1 μg/μl (Straus, 1995, supra).

Genomic subtraction. Genomic subtraction is carried out, as was described previously (Straus, 1995, supra). To isolate pathogen-specific DNA fragments, a genomic subtraction experiment is carried out using the “+” genomic subtraction sample derived from pathogenic strains and the biotinylated “−” genomic subtraction sample derived from non-pathogenic strains. Three cycles of subtractive hybridization purify the pathogen-specific genomic difference sequences.

Cloning the genomic difference sequences. After ligating adaptors to the genomic difference sequences, they are amplified using PCR (Straus, 1995, supra; Straus et al., 1990, supra). The adaptors are then removed from the amplified genomic difference sequences by cutting with Sau3A. The samples are brought to 0.3 M sodium acetate (NaOAc), extracted with phenol/chloroform (1:1), and precipitated with ethanol. A portion of the sample (20 ng) is ligated to BamHI-digested, dephosphorylated vector, pBluescriptII KS+ (100 ng; Stratagene), and the ligated products are transformed into E. coli (Ausubel et al., 1987, supra).

Sequencing the genomic difference products. The inserts of individual clones are sequenced using an ABI DNA synthesizer by cycle sequencing, according to the manufacturer's recommendations (Perkin-Elmer).

Isolating an ensemble of genomic difference sequences from bacteria that cause gastrointestinal illness. By performing genomic subtractions, as is outlined above, on genomic difference samples prepared from organisms in the bacterial groups listed in Table 3, genomic difference sequences from different groups of pathogens that commonly cause gastrointestinal illness are isolated. Each subtraction generates a large number of genomic difference sequences unique to pathogens within a group of strains. For example, a single subtraction between a pathogenic E. coli strain and a non-pathogenic E. coli strain yielded hundreds of genomic difference sequences (Juang, “Sampling Genomic Differences Between Escherichia coli KI ad K12 isolates,” Harvard University, 1990).

Genomic subtraction using DNA sequence databases. Genomic subtraction, in its general sense of scanning whole genomes for genomic difference sequences, can also be achieved by comparing the DNA sequences of a completely sequenced (or nearly completely sequenced) genome with all or part of another genome (or genomes) (see, for example, Alm et al., 1999, supra)).

Preparing Probes and Detection Ensembles Corresponding to the Genomic Difference Sequences

The ensemble of pathogen-specific ID sequences identified, as is described above by genomic subtraction, is used to define the structure of the ID probes that are used in the genomic profiling assay. Two ensembles of ID oligonucleotides are synthesized. One ensemble, constituting the ID probes (or ID probe-halves), is hybridized to a biological sample, ID probe-halves that anneal to pathogenic genomes in the experimental sample are ligated, amplified, and labeled. The other ensemble of ID oligonucleotides constitutes a detection ensemble. The ID oligonucleotides in the detection ensemble correspond to the sequences in the ensemble of ID probes. That is, the detection ensemble is congruent to the ID probe ensemble. The detection ensemble oligonucleotides are deposited onto a solid support, forming an addressable array. The labeled, amplified probes that hybridized to pathogen genomes in the clinical sample are identified by hybridization to the addressable array of oligonucleotides.

Synthesizing ID probes corresponding to the ID sequences. A sequence, referred to as an ID probe site, of approximately 30 bases is chosen from each ID sequence, human mRNA (see below), and control sequence to be included in the genomic profiling assay. Two ID probe-halves are synthesized corresponding to each 30 base ID probe site (FIG. 3). The left ID probe-half contains the left 15 bases of the ID probe site and a primer site, primer site-L (the “left” primer site). The right ID probe-half contains the right 15 bases of the ID probe site and a primer site, primer site-R (the “right” primer site). The primer sites are a type of amplification site that corresponds to the primers to be used for PCR amplification.

The primer site-L (the “left” primer site) has the sequence: 5′-GACACTCTCGAGACATCACCGTCC-3′. The primer site-R (the “right” primer site) has the sequence: 5′-GTTGGTTTAAGGCGCAAGAATT-3′. Thus, for each 30 base sequence identified in the sections above, two ID probe-halves are synthesized: one with the sequence 5′-GACACTCTCGAGACATCACCGTCC-<ID probe site₁₋₁₅>-3′, and one with sequence 5′-<ID probe site₁₆₋₃₀>-GTTGGTTTAAGGCGCAAGAATT-3′. The ID probe-halves are designed so that they abut each other when annealed to a template containing the 30 bp ID probe site. When annealed in this way, the probe-halves can be ligated, and thus converted into a form that can be amplified using primers L (5′-GACACTCTCGAGACATCACCGTCC-3′) and R (5′-AATTCTTGCGCCTTAAACCAAC-3′), which correspond to the left and right primer sites, respectively.

Constructing a detection array for the genomic profiling assay. To determine which probe-halves hybridize to a clinical sample, an addressable detection ensemble of ID sequences can be queried by hybridization. The elements of the ensemble are synthetic ID sequence oligonucleotides that correspond to the ID probe sites in the ensemble of ID probes. That is, each detection oligonucleotide is ˜30 bases long and is complementary to one strand of the ID probe site sequences that result from ligation and amplification of a pair of ID probe-halves.

In this example, I construct a two-dimensional detection array, following the procedure of DiRisi et al. (Science 278:680-686, 1997), using an arraying machine with a printing tip to spot each oligonucleotide (Shalon et al., Genome Res. 6:639-645, 1996). Approximately 2.5 ng of each 30 base oligonucleotide is spotted onto each of 40 slides that have been coated with poly-L-lysine at a spacing of 500 μm between neighboring oligonucleotide spots (Schena et al., 1995, supra).

Constructing a Genomic Profiling Database of Fingerprints

Genomic profiling identifies a pathogen in a patient sample by comparing the genomic profiling fingerprint of the sample to a database containing fingerprints of known organisms. (A fingerprint corresponds to the sub-set of the ensemble of ID probes that hybridizes to a particular type of organism). Constructing a database of fingerprints requires obtaining genomic profiling fingerprints from a set of reference strains from each target group.

Constructing the database is best thought of in terms of the two diagnostic categories into which target groups fall. Most identification schemes fall into two classes (depending on the target group): those that simply test for membership in a group and those that test for membership in a group and distinguish members of a group from each other.

Entering fingerprints composed primarily of group-specific sequences in the database of fingerprints. When membership in a group is the prime consideration, I include primarily group-specific sequences in the family of ID sequences chosen to identify the target organisms. Testing for the presence of a pathogen that is a member of a group (without distinguishing between members of the group) is often the optimal diagnostic strategy when the presence of a member of the group is almost always correlated with disease and when epidemiological information is not of great value. For example, for identifying Vibrio cholerae, a dangerous and virulent gastrointestinal pathogen that causes the life-threatening disease cholera, a family of ID sequences composed mostly of group-specific sequences might be included in the ensemble. Note that the group-specific sequences can be isolated by genomic subtraction in which the “+” strain(s) are pathogens and the “−” strains are non-pathogens. Such ID sequences are both genomic difference sequences and group-specific sequences. Potential group-specific sequences are tested for their specificity by hybridization of each sequence to genomic DNA from representative members of the group and to members of a broad spectrum of other groups (see, for example, U.S. Pat. No. 5,714,321). Thus, an experimental sample that produces a genomic profiling fingerprint composed of positive signals corresponding to group-specific ID sequences indicates the presence of a member of the target group in the sample. Such fingerprints are included in the database of fingerprints.

Entering fingerprints composed primarily of genomic difference sequences in the database of fingerprints. For certain types of organisms, the diagnostic goal may be to identify a strain as a member of a group and at the same time distinguish it from other strains in the group. Sub-strain identification is important, for example, in tracking hospital-acquired infection outbreaks and outbreaks of food-borne pathogens. This type of high-resolution identification requires a more detailed fingerprint than simply identifying a pathogen as a member of a target group (as described in the previous paragraph). Genomic difference sequences isolated by genomic subtraction are the most useful ID sequences for obtaining high-resolution fingerprints.

To construct a database of fingerprints from the target group, I obtain fingerprints from a set of reference strains that are representative of the group. To generate a fingerprint, the genomic profiling assay is applied to a sample (often a single bacterial colony) containing the genome of a single reference strain. The genome is scanned for the presence of members of one or more families of ID sequences (usually genomic difference sequences corresponding to genomic subtraction products) that are characteristic of the target group. The fingerprints obtained are stored in the database. Standard analysis is used to establish the phylogenetic relationship of the reference strains based on the fingerprints (Hillis et al. Molecular Systematics (Sinauer Associates, Sunderland, 1996)).

Constructing databases for high-resolution fingerprinting of food-borne pathogens, such as E. coli O157:H7, is an important tool for tracking outbreaks. For example, I build a database of fingerprints representative of the spectrum of organisms in the E. coli/Shigella group by obtaining genomic profiling fingerprints of reference collections of E. coli and Shigella strains. A large number of such strains are available from the Centers for Disease Control and the American Type Culture Collection. A phylogeny (i.e., evolutionary tree of relatedness) of the group is constructed using the fingerprints as character sets. A powerful feature of this approach is that the fingerprint database for the group becomes progressively more comprehensive as it is updated with new fingerprints of related pathogens discovered in clinical samples.

Preparing a bacterial strain for fingerprinting using the genomic profiling assay. To obtain a fingerprint, I first affix a bacterial colony to a nylon filter and make the genomic DNA of the colony available for hybridization to a probe using a simple and standard method (Grunstein et al., 1975, supra). The colony is smeared on a nylon filter (1 cm²), allowed to dry, and treated successively (for 5 minutes each) with 0.5 M NaOH, 1 M Tris, pH 8/3 M NaCl, 1 M Tris, pH 8. The sample, now fixed to the nylon filter, is washed 3 times for 5 minutes times in 1 M NaCl at 65° C., with shaking, to remove non-fixed chemical and particulate matter. Efficient lysis of some bacteria (and other organisms) may be enhanced by pre-treating the smeared organisms on the filter with specific enzymes or chemicals before the alkaline treatment. For example, lysis of gram positive bacteria is aided by treating filters with a solution containing phospholipase and lysozyme (Graves, L, et al. (1993), “Universal bacterial DNA isolation procedure,” In Diagnostic Molecular Microbiology, Principles and Applications, D. Persing et al., eds. (Washington, D.C. ASM Press), pp. 617-621).

Selecting the subset of genomic difference sequences that hybridize to the DNA of a bacterial strain. The genomic profiling assay selects for the subset of pathogen-specific ID probes that hybridizes to the genomic DNA bound to the nylon filter. In contrast, genomic difference probes that have no counterpart in the fixed bacterial DNA are easily removed from the filter. Any residual ID probe-halves that remain affixed to the filter by non-specific interactions with the filter or sample will not be rendered amplifiable during the subsequent ligation step.

A set of probe-halves (1 nM, each probe-half) corresponding to the pathogen-specific genomic difference sequences derived from a particular group of bacteria are hybridized to the filter at 36° C. (or 5° C. less than the lowest Tₘ of all the half-probes in 1 M NaCl) in 0.5 ml hybridization buffer (1 M NaCl/50 mM EPPS/2 mM EDTA, pH 8). The hybridization reaction is incubated for 30 minutes, after which the unbound probe-halves are removed by five 30 second washing steps, with shaking, at 36° C. (or 5° C. less than the lowest Tₘ of all the half-probes in 1 M NaCl) in 2 ml wash buffer (1 M NaCl/50 mM EPPS/2 mM EDTA, pH 8). The filter is next washed 3 times successively at 30° C. with 1 ml ligation buffer (10 mM MgCl₂/50 mM Tris-HCl/10 mM dithiothreitol/1 mM ATP/25 μg/μl bovine serum albumin). Excess liquid is removed from the filter before proceeding to the ligation step. The filter is not permitted to dry between steps.

Ligating pairs of probe-halves that hybridize to the bacterial sample. Eliminating background due to non-specifically bound probe molecules is critical for the genomic profiling assay, especially as applied below to clinical samples, since high sensitivity is required to detect uncultured pathogens in such samples, as is described in the next section. Recall that requiring ligation of adjacently bound probe-halves is an effective way to insure that the only probes that can be amplified are those that have hybridized to pathogen genomes in the sample.

Probe-halves hybridized to the fixed sample are ligated by adding 200 μL of ligase buffer (10 mM MgCl₂/50 mM Tris-HCl/10 mM dithiothreitoul mM ATP/25 μg/μl bovine serum albumin) containing 1,600 cohesive end units (equivalent to 25 Weiss units) of T4 DNA ligase (New England Biolabs). The ligation reaction is allowed to proceed for 1 hour at 30° C.

Amplifying the genomic difference sequences that hybridize to the bacterial sample. Pairs of ligated probe-halves that hybridize to the genomes in the bacterial sample are released from the filter by heating. The ligated probe-halves are then amplified using the polymerase chain reaction and primers corresponding to the primer binding sites at the ends of the ligated probe molecules.

After ligation of the probe-halves, filters are washed with 2 ml 10 mM EPPS/1 mM EDTA, pH 8.0, the liquid is removed from the filter, and 500 μl of 10 mM EPPS/1 mM EDTA, pH 8.0 is added to the filter, which is then incubated for 5 minutes at 100° C. After separating the solution from the filter, 50 μl 3 M sodium acetate and 20 μg yeast tRNA are added. The nucleic acids are purified by ethanol precipitation: 1 ml of ethanol is mixed with the sample, after which the sample is centrifuged at 12,000 g for 5 minutes. The nucleic acid pellet is washed with 100% ethanol, dried, and resuspended in 10 μl 10 mM EPPS/1 mM EDTA, pH 8.0.

Half (5 μl) of the sample containing the eluted probe is brought to 1×PCR buffer using 10×PCR buffer (Boehringer Mannheim), 200 μM of each dNTP (dATP, TTP, dCTP, and dGTP), 1 μM biotinylated oligonucleotide primer L (5′-(biotin-dX)GACACTCTCGAGACATCACCGTCC-3′) (Midland Certified Reagent), 1 μM biotinylated oligonucleotide primer R (5′-(biotindX)AATTCTTGCGCCTTAAACCAAC-3′), and 0.1 unit/μl Taq polymerase (Promega), in a total reaction volume of 50 μl. The eluted probes are amplified using a PCR regime of 30 cycles (30 seconds at 94° C., 30 seconds at 55° C., and 1 minute at 72° C.), followed by 10 minutes at 72° C.

The genomic profiling fingerprint of a strain: identifying the amplified probe molecules selected by the bacterial DNA by hybridization with an array. A fingerprint of a strain is established by identifying the ID probes that are selected by hybridization to the immobilized DNA of the strain. In this example, I identify the ID probes selected by the bacterial genomic DNA by hybridizing the amplified, selected ID probes to a detection array. The detection array is a two-dimensional addressable array of sequences, congruent to the ensemble of ID probes used to hybridize to the biological sample. Thus, each ID probe in the ensemble can hybridize to a DNA sequence at a defined site on the detection array. The probes selected by binding to the bacterial sample are identified by hybridization to the array. Only the selected probes generate signals by binding to the corresponding spots on the array (FIG. 5).

I denature half (25 μl) of the amplified probe, representing the sequences that hybridized to the bacterial sample, by heating at 100° C. for 1 minute. The denatured probe is added to 25 nml of 2× hybridization solution (2 M NaCl/100 mM EPPS, pH 8/10 mM EDTA/0.2% Sodium Dodecyl Sulfate). The probe/hybridization mixture is placed on the array, covered with a glass coverslip, and incubated for 20 minutes at 50° C. (as described in Schena et al., 1995, supra). The unbound probe is removed by five 30 second washing steps, with shaking, at 50° C. in 2 ml wash buffer (0.4 M NaCl/50 mM EPPS/2 mM EDTA, pH 8).

Microarrays are scanned with a laser fluorescent scanner, and signals are processed and recorded as is described in published reports (DiRisi et al., 1997, supra; Schena et al., 1995, supra). The fingerprint of each strain is recorded as a binary string of l's and 0's, with each digit representing one genomic difference sequence on the microarray. If a signal is obtained at a site on the microarray, a “1” occurs at the corresponding digit in the string representing the genomic profiling fingerprint.

Using genomic profiling fingerprints and phylogenetic analysis for typing strains in a group. The fingerprint database for representative strains in a group is useful for identifying unknown strains. A database of fingerprints is compiled as is described above, and phylogenetic analysis of the fingerprints is performed using standard methods, as are described in Hillis et al., supra. The identity of an unknown pathogen, for example, one in a patient sample, is determined by comparing the unknown fingerprint to the phylogenetically ordered database of fingerprints (using methods described in Hillis et al., supra).

Isolating ID sequences from parasites that cause gastrointestinal illness Parasites that cause gastrointestinal illness. The spectrum of intestinal parasites found in patients varies, depending on geographical location, climate, socioeconomic factors, and immunological competence. Table 3 lists groups of protozoa and helminths that are commonly found in patients with gastrointestinal illness in North America. Current methods for accurate diagnosis of intestinal parasites are difficult, at best. Genomic profiling greatly improves the detection of gastrointestinal parasites.

Isolating ID sequences from parasites that cause gastrointestinal illness. To isolate sets of ID sequences that are unique to each parasite in Table 3, I use the same strategy and methodology outlined above for bacterial pathogens, with the following small modifications. Because parasites are generally not related to organisms normally found in the gut, it usually suffices to construct the genomic difference samples from the genomic DNA of two strains that are most widely separated within the taxon of interest. Reciprocal subtractions are carried out, i.e., each strain serves as the “+” strain in one subtraction and the “−” strain in the other subtraction. Increasing the incubation times for the subtractive hybridization reactions, relative to the incubation time for the bacterial subtractions, is necessary to compensate for the increased complexity of eukaryotic genomes. I use reassociation times of forty to fifty times the time required for half of the single copy sequences to reanneal (Straus, 1995, supra).

Constructing a database of parasite fingerprints. As is described above for fingerprinting bacterial pathogens, the parasite ID sequences are used to construct families of ID probes for identifying the organisms listed in Table 3. Fingerprinting reference strains and constructing a database of fingerprints is also carried out as is described for the bacterial pathogens.

Identifying Group-Specific Sequences of Viruses that Cause Gastrointestinal Illness

Viruses that cause gastrointestinal illness. Viral gastroenteritis is thought to be the second most common cause of illness in the United States. Children are particularly susceptible, as are immunocompromised patients. Diagnosing virus-caused gastrointestinal illness is problematic, as most of the common agents are not culturable and are poorly characterized. The tests that have been developed are generally very expensive. Diagnostic tests are generally not done due to the expense of the available tests, the infrequency of serious complications, the common supportive treatment, and the lack of anti-viral therapies. However, comprehensive and inexpensive test for viruses will be useful for epidemiology, for ruling out other causes, for ruling out use of antibiotics, and for indicating appropriate administration of new anti-viral therapies. Table 3 lists viral pathogens that commonly cause gastrointestinal illness.

Identifying group-specific sequences from viruses that cause gastrointestinal illness. For viruses that cause gastrointestinal illness, group-specific ID sequences are deduced from published DNA sequence data. In some cases, viral group-specific sequences are already described in the literature. In other cases, sequences are chosen from viral genomic sequences in public databases after comparing the sequences to other viruses in the database. Sequence comparisons are made using standard methods (Ausubel et al., 1987, supra). Viral group-specific sequences that are at least 30 bp are chosen as targets for assay probes.

Constructing a database of viral fingerprints. As is described above for fingerprinting bacterial pathogens, the parasite ID sequences are used to construct families of ID probes for identifying the viruses in Table 3. Fingerprinting reference viral strains and constructing a database of viral fingerprints is also carried out as is described for the bacterial pathogens, except for the sample preparation. For viruses containing RNA genomes, the sample preparation must ensure the integrity of the RNA. I process the filters by autoclaving (Allday et al., Nucleic Acids Res. 15:10592, 1987) or baking in a microwave oven (Buluwela et al., Nucleic Acids Res. 17:452, 1989) to denature the genomic nucleic acid, fix it to filters, and make it accessible to probes.

Human Sequences Useful in Diagnosing Gastrointestinal Illness

An advantage to the genomic profiling assay is that diagnostically useful human cell types can be assayed in the same test that screens for pathogens. For example, in gastrointestinal illness it is important to know whether leukocytes and erythrocytes are over-represented in a clinical sample. To test for specific cell types, sequences of cell type-specific mRNAs are obtained (generally from published reports or genetic databases). Table 4 indicates cell-type specific mRNAs of known sequences that are expressed in certain cell types and are important in diagnosing gastrointestinal illness.

Probes analogous to ID probes are synthesized (i.e., as binary probe-halves with amplification sites) and are included in the hybridization mixture used to contact the prepared biological sample. The corresponding detection sequences are included on the detection array.

TABLE 4
Probes for Human Cells Important for Diagnosing
Gastrointestinal Illness.

| transcript | characteristics of transcript |
|---|---|
| Lactoferrin | Product of white blood cells - indicative of invasive |
| | infection |
| LCA, CD45 | Leukocyte specific |
| Globin | Product of red blood cells - indicates bleeding |
| Actin | Common to all human cells (use as human-specific probe) |

Internal Control Sequences Useful for Evaluating the Genomic Profiling Assay

Internal controls. Including internal controls in the genomic profiling assay improves confidence in the test results and allows efficient troubleshooting. Control probes, oligonucleotides, and detection sequences contain non-biological sequences.

Positive control sequences give a positive signal in every experiment if the technique is working. If, for example, one of the reagents is not functioning properly, the expected signal from the positive control is absent. The missing signal from the positive control ensures that false negatives, due to technical failure are avoided.

Negative controls are included to monitor whether sequences in the probe that are not in the clinical sample are causing signals on the diagnostic detection array. The genomic profiling assay is designed so that signals should only be obtained on the detection array if an ID probe in the ID probe ensemble corresponds to an ID sequence in the clinical sample. The deployment of the negative controls is similar to the positive control, except that no corresponding sequences are spotted with the clinical sample (i.e., it is included in the hybridization mixture with the ID probe ensemble and is an element of the detection array). Thus, the negative control sequence should not be capable of being selected by the fixed sample, ligated, or amplified. A positive signal from the negative control sequence in the detection array indicates that the steps that select for hybridization of ID probes with target sequences are not working adequately.

I include another control probe in the assay that allows monitoring of the ligase reaction. This probe is synthesized, not as a probe-half, but as a continuous sequence tagged with both left and right adaptors. Otherwise, the sequence is used as the positive control probes (i.e., it is spotted in parallel to the clinical sample, it is included in the probe, and is an element of the detection array). If the positive control element of the detection array is negative, but the ligase control element of the detection array is positive, the ligase step in the assay is suspect.

TABLE 5
Internal controls for genomic profiling assay.

| | | control | |
|---|---|---|---|
| | | sequence | control |
| | | present | sequece |
| | | on filter | present |
| type of control | function of control | with sample | in probe |
| negative con- | indicates background level of | no | yes |
| trol | signal obtained from probes that | | |
| | do not match DNA in sample | | |
| ligation control | gives positive signal if all non- | yes | yes |
| | ligation steps in assay are | | |
| | working | | |
| positive control | gives positive signal if all steps | yes | yes |
| | in assay are working | | |

Identifying Pathogens Present in a Clinical Sample

Preparation of clinical samples. For genomic profiling to be most effective in a clinical setting, a simple method for preparing clinical samples for hybridization to the probe-halves is preferred. Preparation of the patient sample should also ideally feature rapid neutralization of pathogens present in the sample, for safety of laboratory workers, and should effectively remove inhibitors of subsequent enzymatic reactions, such as probe amplification.

I fix the clinical sample, denature nucleic acid molecules, and neutralize any pathogens with a simple, general, and yet effective method that is commonly used for preparing biochemically complex biological samples for hybridization (Grunstein et al., 1975, supra). A gastrointestinal sample (0.5 ml liquid fecal sample, formed stool sample, or rectal swabs sample) is smeared on a nylon filter (1 cm²), allowed to dry, and treated as is described above for the preparation of viral samples. The sample, now fixed to the nylon filter, is washed several times at 65° C., with shaking, to remove non-fixed chemical and particulate matter.

Scanning a clinical sample for the presence of genomic difference sequences by hybridization. I scan a gastrointestinal sample for the comprehensive set of relevant pathogens by hybridizing the ensemble of ID probes, the human diagnostic sequences, and the control sequences to a clinical sample. The protocol is essentially the same as that used to fingerprint reference strains for building a database of bacterial fingerprints (see above), except for the comprehensive composition of the ID probe ensemble and that a clinical sample (prepared as is described in the previous paragraph) serves as the biological sample.

Obtaining a genomic profiling fingerprint for a clinical sample. The ligation, amplification, and fingerprint development (array detection) follow the same protocol as is detailed above for bacteria (see “Constructing a database of genomic profiling database of fingerprints”), with the exception that the array contains a detection ensemble representing all of the pathogens indicated in Table 3. The detection sequences on the detection array correspond to the ensemble of ID probes, human diagnostic sequences, and control sequences that are hybridized to the clinical sample.

Quantitative analysis: what is the titer of pathogens in the clinical sample? A powerful feature of the genomic profiling assay is the ability to quantify pathogens in a biological sample. Once target organism(s) have been identified by a fingerprint, their presence can be quantified by in situ hybridization to a portion of the original biological sample prepared according to standard methods (e.g., Huang et al., Modern Pathology 11:971-977, 1998). I use a sensitive, yet simple, method that is powerful enough to detect a single molecule of a nucleic acid sequence in a single organism (Huang et al., supra, 1998). This method is used with the labeled probes used for hybridization to the detection array. Alternatively, any other group-specific probes that are diagnostic for the organism(s) detected by hybridization to the array may be used for in situ hybridization.

### EXAMPLE 2

### Testing a Respiratory Sample for the Presence of Pathogens

Pneumonia. Pneumonia is the most common cause of death from infectious disease in the United States. The etiology of the disease is dependent on age and immune status. Viruses cause most childhood pneumonia, while bacterial pathogens are the most common pathogens causing adult pneumonia. The spectrum of pathogens that cause pneumonia in immunocompromised hosts varies greatly and differs for patients with cancers affecting the immune system or protective surfaces (mucosal or skin), transplant recipients, and HIV-infected patients.

For successful treatment of pneumonia, it is essential to rapidly identify the pathogen. Yet, almost half of all diagnostic efforts to determine the cause of pneumonia fail to identify the etiologic agent. (This does not include the large fraction of cases in which no attempt is made to identify the pathogen.) Many bacterial and all viral pathogens that cause lower respiratory tract infections cannot be identified by routine microbiological culture methods. For example, special methods are required to identify the pathogens that cause tuberculosis, whooping cough, legionnaire's disease, and mycoplasma-caused pneumonia. Patients with lower respiratory infections account for 75% of antibiotics prescribed in the United States. Nearly $1 billion a year is wasted on useless antibiotics, due to the failure of current diagnostics to identify the pathogen in most lower respiratory tract infections. Thus, there is a great need for a single diagnostic assay that tests for a comprehensive set of lower respiratory pathogens.

Objectives and Advantages. In this example, I use a single genomic profiling assay to test for the presence of respiratory pathogens in a sample from a patient with symptoms of lower respiratory disease. By simultaneously and rapidly (e.g., in several hours) testing for common bacterial, viral, and protozoan pathogens, the method offers a substantial improvement over current practices. The test helps to determine an appropriate and timely therapy. Furthermore, the genomic profiling assay is a powerful tool for epidemiological analysis, because it can produce high-resolution fingerprints.

Overview of the example. I isolate ID sequences from various lower respiratory tract pathogens using genomic subtraction, in the cases of bacterial pathogens and parasites, or computer analysis, in the case of viruses. The subset of genomic difference sequences that are present in the DNA of a given strain constitutes its genomic profiling fingerprint. A fingerprint database is constructed by determining the subset of ID sequences present in representative strains from each group of respiratory pathogens. The identity of pathogens in a clinical respiratory sample is determined by comparing the genomic profiling fingerprint of the clinical sample to the database of fingerprints.

Overview of the methods used in the example. In this example, I use suppression subtractive hybridization to isolate pathogen-specific genomic difference sequences, rather than the genomic subtraction method used in Example 1. As in the previous example, determining the identity of the ID sequences in a particular sample is accomplished by using the genomic DNA of the sample to select, by hybridization, a set of ID probes. The selected ID probes are then amplified using the hyperbranched rolling circle amplification method (HRCA) (Lizardi et al., Nat. Genet. 19:225-232, 1998). I determine the identity of the ID probes selected by the sample by using a different detection array technology than the one described in Example 1.

Isolating ID sequences from pathogens that cause lower respiratory disease. Table 6 lists some common pathogens that cause lower respiratory infections, ID sequences are isolated from the non-viral (i.e., bacterial and fungal) pathogens using a suppression subtractive hybridization kit from Clontech (Diatchenko et al., Proc. Natl. Acad. Sci. USA 93:6025-6030, 1996), according to the manufacturer's recommended protocol. Choosing a subtraction scheme (e.g., the choice of using pooled genomic difference samples vs. single-strain genomic difference samples) for isolating ID sequences from the various groups is the same as is Example 1. As is described in Example 1, the “+” genomic difference sample for a particular group listed in Table 6 is composed of DNA from one or more representative pathogens from the group, while the “−” strain is composed of DNA from one or more closely related, non-pathogenic organisms. (For groups in which all known representatives are pathogens, the “+” and “−” samples include pooled DNA from subgroups of pathogenic strains.) Genomic difference sequences isolated by genomic subtraction are sequenced in preparation for synthesis of rolling circle amplification probes and primers (see below).

For viruses that cause lower respiratory illness, group-specific sequences are deduced from published DNA sequence data, ID probes are synthesized that correspond to sequences that are conserved within a group of viruses, but that are not found in other viral groups. I choose sequences that fulfill the comparative criteria by comparing potential group-specific sequences to viral sequence databases (e.g., Genbank).

TABLE 6
Pathogens that cause lower respiratory illness.

Bacteria
Cornybacterium diphtheriae
Mycobacterium tuberculosis
Mycoplasma pneumoniae
Chlamydia trachomatis
Chlamydia pneumoniae
Bordetella pertussis
Legionella spp.
Nocardia spp.
Streptococcus pneumoniae
Haemophilus influenzae
Chlamydia psittaci
Pseudomonas aeruginosa
Staphylococcus aureus
Fungi
Histoplasma capsulatum
Coccidoides immitis
Cryptococcus neoformans
Blastomyces dermatitidis
Pneumocystis carinii
Viruses
Respiratory syncytial virus
Adenovirus
Herpes simplex virus
Influenza virus
Parainfluenza virus
Rhinovirus

Tissue-specific sequences useful for judging the quality of a respiratory sample. Respiratory samples are notorious for being of uneven quality. Sputum samples, which are conveniently and non-invasively collected, are frequently rejected because of contamination by organisms of the upper-respiratory tract. Systems for judging the quality of specimens have been developed based on the microscopically observed ratio of squamous epithelial cells to polymorphonuclear leucocytes. I include, in my respiratory assay, an internal hybridization-based test for judging the quality of a lower respiratory tract sample based on the relative abundance of these two cell types. This is accomplished by testing for the relative levels of transcripts from cell-type specific transcripts from polymorphonuclear leukocyte (encoding the proteins LCA and CD45) and squamous epithelial cells (encoding the protein spr 1).

Tissue-specific sequence probes are synthesized with probe sites that correspond to the tissue-specific sequences using the same methods used for constructing ID probes corresponding to ID sequences, except that the sequences are obtained from the GenBank database. These probes are included in the hybridization mixture with the ensemble of ID probes and on the detection array.

Included too are control sequences for quantifying the representation of the tissue-specific mRNAs. The control sequences are a series of distinct non-biological RNA sequences that are added to the biological sample in various amounts. The corresponding probes and detection sequences are included in the hybridization mix and detection array. Calibration of these quantitative controls is accomplished by performing the assay on samples with known numbers of squamous epithelial cells and polymorphonuclear leukocytes.

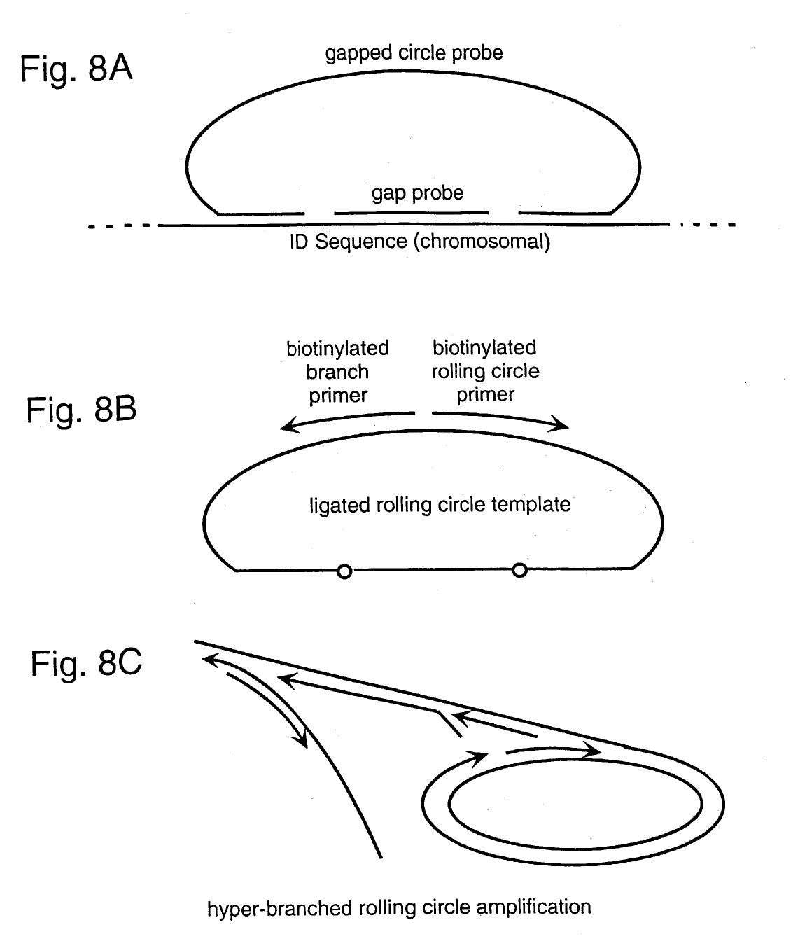

ID probes and primers for rolling circle amplification. For each ID sequence in the respiratory genomic profiling assay, a pair of ID probes (FIG. 8A) and a pair of primers (FIG. 8B) are synthesized, ID probes and primers are based on those in the gap oligo method of Lizardi et al. (1998, supra). However, the gap ID probe (˜15 bases) and the ends of the gapped circle ID probe (˜15 bases) correspond to an ID sequence. Also, in this example, I use 5′ biotinylated primers for rolling circle amplification (FIG. 8C). Similarly, ID probes are synthesized corresponding to the experimental control sequences described in Example 1 and to tissue specific RNAs.

Constructing two-dimensional detection arrays for the genomic profiling assay. To determine which ID probes hybridize to a sample, I hybridize the amplified selected ID probes to a detection array (an addressable array comprising an ensemble of detection sequences). The elements of the array include oligonucleotides that correspond to the gap probe moiety of rolling circle amplification probe pairs or to experimental control sequences. In this example, I construct microarrays using photolithography, as was described previously (Chee et al., Science 274:610-614, 1996; Lockhart et al., Nat. Biotech. 14:1675-1680, 1996).

Fingerprinting Respiratory Pathogens

To identify a pathogen that is the cause of a lower respiratory tract infection, I compare the genomic profiling fingerprint of a clinical sample to a database of fingerprints from previously characterized organisms. As in Example 1, which relates to a gastrointestinal genomic profiling assay, I first assemble the fingerprint database from the genomic profiling fingerprints of reference strains from each group of pathogens. The fingerprints of a clinical sample are then compared to the database to determine the identity of pathogens in the sample.

Obtaining fingerprints of a reference strain and assembling a database. Sample preparation, hybridization to the ensemble of ID probes, and washing steps are identical to those described in Example 1, except for the composition and structure of the ensemble of ID probes. Templates for hyperbranched rolling circle amplification (HRCA) are created when pairs of gapped circle ID probes and gap ID probes that anneal with DNA in the fixed sample are ligated to each other. Ligation and HRCA are carried out as is illustrated in FIG. 8 and as was described previously (Lizardi et al., 1998, supra). Hybridization to the microarray, staining using streptavidin-phycoerythrin, and scanning are accomplished as was described previously (Lockhart et al., 1996, supra), Fingerprints are obtained from the microarray data and a database of fingerprints from each group of respiratory pathogens is assembled and analyzed using the methods described in Example 1.

Identifying pathogens present in a clinical sample. Various types and qualities of respiratory samples (e.g., sputum, bronchoalveolar lavage, and bronchial brush samples) are applied and fixed to nylon filters using the method described in Example 1. As in Example 1, clinical samples are fingerprinted, as are reference strains, except that the ID probes from all of the respiratory pathogens groups in Table 6 are included in the hybridization reaction. Pathogen(s) present in a clinical sample are identified by comparing the fingerprint(s) obtained to those in the database of fingerprints of reference strains.

### EXAMPLE 3

### Testing Blood Samples for Pathogens

Bloodstream infections. Pathogenic invasion of the cardiovascular system is one of the most serious infectious diseases. Of the approximately 200,000 bloodstream infections that occur every year in the United States, between 20 and 50 percent are fatal. Particularly at risk are immunocompromised patients, the very young and very old, those with skin or soft tissue infections and wounds, and the recipients of invasive medical procedures. All major types of pathogens can infect the bloodstream, including bacteria, viruses, fungi, and parasites. Rapid identification of a pathogen in a bloodstream infection is critical for instituting appropriate, potentially life-saving, therapy.

Current methodologies are generally pathogen-specific. Consequently, many tests and much expense can be required to determine the source of infection. There is a need for a single assay that rapidly determines the identity of a broad range of common bloodstream pathogens.

Objectives and Advantages. In this example, I use a single genomic profiling assay to test for the presence of a broad range of bloodstream pathogens in a clinical sample. By simultaneously and rapidly (e.g., in several hours) testing for common bacterial, viral, and protozoan pathogens, the method offers a substantial improvement over current practices. The rapidity of the test makes it particularly useful for the critical task of quickly diagnosing bloodstream pathogens and for instituting appropriate and timely therapy. Furthermore, the genomic profiling assay is a powerful tool for epidemiological analysis, because it can produce high-resolution fingerprints.

Overview of the example. I isolate ID sequences from various bloodstream pathogens using genomic subtraction (bacterial pathogens and parasites) or computer analysis (viruses). The subset of ID sequences that are present in the DNA of a given strain constitutes its genomic profiling fingerprint. A fingerprint database is constructed by determining the subset of ID sequences present in representative strains from each group of bloodstream pathogens. The identity of pathogens in a clinical bloodstream sample is determined by comparing the genomic profiling fingerprint of the clinical sample to the database of fingerprints.

Overview of the methods used in the example. In this example, I use the modified representational difference analysis genomic subtraction method of Tinsley et al. (Proc. Natl. Acad. Sci. USA 93:11109-11114, 1996) to isolate pathogen-specific ID sequences, rather than the methods used in the previous examples. As in the previous examples, determining the identity of the ID sequences in a particular sample is accomplished by using the genomic DNA of the sample to select by hybridization a set of ID probes. In this example, however, the selected probes are isolated by a solution phase hybridization-capture method. Also, in this example, I identify the selected, amplified ID probes using mass spectrometry, rather than by using the microarray methods described in the previous examples.

Isolating ID sequences from pathogens that cause bloodstream infections. Table 7 lists some common pathogens that cause bloodstream infections, ID sequences are isolated from the non-viral (i.e., bacterial, fungal, and parasitic) pathogens using the representational difference analysis method, as modified by Tinsley et al. (1996, supra). As is described in Example 1, the “+” genomic difference sample for a particular group listed in Table 7 is composed of DNA from representative pathogens from the group, while the “−” genomic difference sample is composed of DNA from closely related, non-pathogenic organisms. (For groups in which all known representatives are pathogens, the “+” and “−” samples include pooled DNA from subgroups of pathogenic strains.) For viruses that cause bloodstream infections, ID sequences are deduced from published DNA sequence data, as is described in the previous examples.

TABLE 7
Pathogens that cause bloodstream infections.

Bacteria
Coagulase-negative staphylococci
Staphylococcus aureus
Viridans streptococci
Enterococcus spp.
Beta-hemolytic streptococci
Streptococcus pneumoniae
Escherichia spp.
Klebsiella spp.
Pseudomonas spp.
Enterbater spp.
Proteus spp.
Bacteroides spp.
Clostridium spp.
Pseudomonas aueruginosa
Cornybacterium spp.
Fungi
Plasmodium spp.
Leishmania donovani
Toxoplasma spp.
Microfilariae
Fungi
Histoplasma capsulatum
Coccidoides immitis
Cryptococcus neoformans
Candida spp.
Viruses
HIV
Herpes simplex virus
Hepatitis C virus
Hepatitis B virus
Cytomegalovirus
Epstein-Barr virus

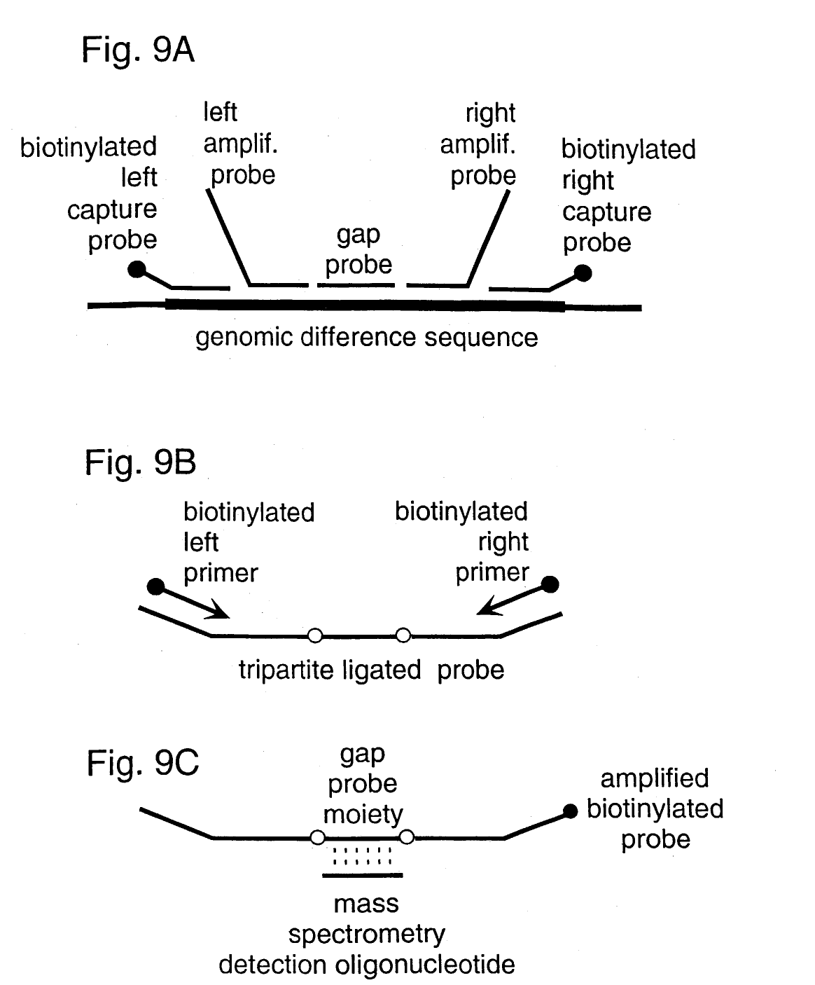

ID probes for capturing ID sequences, amplification, and mass spectrometry detection. For each ID sequence in the bloodstream genomic profiling assay, a pair of DNA capture ID probes, two amplification ID probes, a gap ID probe, and one mass spectrometry detection oligonucleotide are synthesized (FIGS. 9A-9C). Each capture ID probe has two moieties: a biotinylated arm (approximately 10 bases long) and an arm that corresponds to a section of an ID sequence (approximately 15 bases long). The left and right amplification probes also have two moieties: one part contains a sequence corresponding to an amplification primer (about 20 bases long) and one part is complementary to an ID sequence (about 15 bases long). Primers, biotinylated on the 5′ end, are synthesized so that the ligated tripartite probe can be amplified (FIG. 9B) and affinity purified. The gap ID probe (approximately 20 bases long) is complementary to an ID sequence and abuts the left and right amplification ID probes when annealed to the corresponding ID sequence. Positive and negative control probes are synthesized and employed similarly to those described in Example 1, except that positive control sequences that are bound to the filter in Example 1 are included in the sample solution in this Example.

To determine which ID probes hybridize to a sample, I hybridize the amplified, selected ID probes to an ensemble of mass spectrometry detection oligonucleotides that are congruent to the ensemble of ID probes being assayed. Each mass spectrometry detection oligonucleotide is approximately 8-15 nucleotides long (mass spectrometry achieves very high resolution discrimination of small oligonucleotides), and each is complementary to the gap probe moiety of one ID probe (FIG. 9C). The individual mass spectrometry detection oligonucleotides in the ensemble should all have distinct molecular weights, such that their identity can be determined by mass spectrometry. To enhance the molecular weight differences between oligonucleotides with similar molecular weights, in certain cases, it is useful to include chemically modified oligonucleotides. Oligonucleotides with a great variety of chemical modifications and with minimally altered reassociation characteristics are commercially available.

Fingerprinting Bloodstream Pathogens

As in the previous examples, to identify a pathogen that is the cause of a bloodstream infection, I compare the genomic profiling fingerprint of a clinical sample to a database of fingerprints from previously characterized organisms. As before, I first assemble the fingerprint database from the genomic profiling fingerprints of reference strains from each group of bloodstream pathogens listed in Table 7. The fingerprint of a clinical blood sample is then compared to the database to determine the identity of any pathogens in the sample.

Capturing and amplifying ID probes that hybridize to the DNA of a reference strain. In this example, I use a solution phase hybridization-capture method (Hsuih et al., J. Clin. Microbiol. 34:501-507, 1996) to affinity purify pathogen-specific ID sequences that are present in the nucleic acid molecules of a reference strain. Organisms are lysed and nucleic acid molecules of the organism are made available for hybridization by incubation in 5 M guanidine thiocyanate (5 minutes at 90° C., followed by 10 minutes at 65° C.) and by vortexing briefly. Depending on the organisms to be detected, this procedure can be modified by, for example, including heat treatment at a higher temperature, enzymatic treatment (e.g., with lysozyme, chitinase, or phospholipase), treatment with a detergent (e.g., CTAB or SDS), or organic extraction (e.g., With phenol or chloroform). I then follow the method of Hsuih et al. (1996, supra) for hybridization with probes (capture, amplification, and gap), affinity purification, ligation, and amplification of the tripartite ligated amplification/gap probe (FIG. 9B) (Hsuih et al., 1996, supra).

Purifying mass spectrometry detection oligonucleotides corresponding to the amplified ID probes. The amplified probes correspond to pathogen-specific ID sequences in the reference strain. For mass spectrometric-based identification of these sequences, I use the biotinylated amplification products to affinity purify the corresponding mass spectrometry detection oligonucleotides (FIG. 9C). Amplification reactions (50 μl) are brought to 10 mM EDTA, combined with a 10 μl of a solution containing 10 ng of each mass spectrometry detection oligonucleotide in 10 mM EPPS, pH 8.0/1 mM EDTA, and denatured at 100° C. for 2 minutes. After adding 15 μl 5 M NaCl and incubating for 15 minutes at 30° C., 30 μl of streptavidin-coated paramagnetic beads (Promega) are added and affinity chromatography is carried out as was described previously (Hsuih et al., 1996, supra). The beads are washed 3 times with 500 μl 10 mM EPPS, pH 8.0/1 mM EDTA. Affinity purified mass spectrometry detection oligonucleotides are recovered by heating the solution to 50° C. in 100 μl 10 mM EPPS, pH 8.0/1 mM EDTA (or 10° C. higher than the highest Tₘ of the detection oligonucleotides in 1 M NaCl). The supernatant containing the mass spectrometry detection oligonucleotides is removed from the magnetic beads, which are retained in the tube using a magnet.

Constructing a database of fingerprints for a group of pathogens: using mass spectrometry to identify the selected mass spectrometry detection oligonucleotides. Samples are prepared and analyzed by matrix-assisted laser desorption ionization time-of-flight mass spectrometry (delayed extraction) (MALDI-TOF (DE)) using the instrument (PerSeptive Biosystems) and methods described previously (Roskey et al., Proc. Natl. Acad. Sci. USA 93:4724-4729, 1996). The masses of the affinity purified oligonucleotides are compared to the previously determined masses of the elements of the entire ensemble of mass spectrometry detection oligonucleotides. In this way, the selected mass spectrometry detection oligonucleotides are identified, which in turn indicates the identities of the ID sequences in the reference strain being tested.

The subset of ID sequences present in the reference strain constitutes its genomic profiling fingerprint. A database of fingerprints is collected for reference strains in each group listed in Table 7.

Identifying pathogens present in a blood sample. Blood samples (1 ml) are lysed and fingerprinted as is described above for the reference strains, except that the ID probes from all of the bloodstream pathogen groups in Table 7 are included in the hybridization reaction. Pathogen(s) present in a blood sample are identified by comparing the fingerprint(s) obtained to those in the database of fingerprints of reference strains.

### EXAMPLE 4

### Forensic Identification Using the Genomic Profiling Assay

Overview of forensic identification. Identifying the origin of cellular samples is a critical aspect of modern medico-legal analysis. Genetic identification of forensic samples requires that DNA in cellular material, often available in only microscopic quantities, be amplified and compared to that of other individuals. Current methodologies for genetic identification generally require analytical gel electrophoresis, which is time consuming and technically unsuited for many forensic laboratories. This example provides a rapid, simple, and robust method for forensic identification using genomic profiling.

Overview of the example. I isolate an ensemble of ID sequences that are useful for identifying the origin of human forensic samples using enriched genomic difference samples. In this example, the enriched genomic samples are amplified subsets of human genomes which, by the nature of the amplification process, contain some sequences that are reproducibly amplified from the genomes of some individuals but not from those of other individuals. These differentially amplified sequences constitute genomic difference sequences: they are present in one enriched genomic difference sample but not another. The subset of an ensemble of such sequences that are present in the DNA from an individual constitutes a genomic profiling fingerprint. The identity of the source of the sample can be obtained by comparing the sample fingerprint to that of other individuals.

Overview of the methods used in the example. This example differs from the previous examples in several ways. The enriched genomic difference samples used to obtain the ensemble of human ID sequences are constructed by selectively amplifying human genomic DNA. This example selectively amplifies human DNA using Alu-PCR, but other methods can also be used for selective amplification, such as the AFLP method, methods that amplify size fractionated DNA (Lisitsyn et al. Mol. Gen. Microbiol. Virus. 3:26-29, 1993; Rosenberg et al., Proc. Natl. Acad. Sci. USA 91:6113-6117, 1994), or the method described in Example 5. Multiple genomic subtractions are carried out to generate numerous families of human ID sequences. Detection sequences corresponding to genomic subtraction products are used to construct a detection array. To identify a human forensic sample, the sample DNA is amplified using selective amplification (in this case Alu-PCR). The resulting “representation” of the human genomic DNA in the sample is composed of labeled amplification products. The products are tested for the presence of diagnostic ID sequences by hybridization to the detection array. The genomes of different human individuals will generate different genomic profiling fingerprints.

Selective amplification of human DNA using Alu-PCR. The Alu-PCR method amplifies DNA between Alu repeats, which occur frequently in the human genome (every few thousand bases, on average). Because Alu repeats are polymorphic, some amplified fragments are present in one person, but not another (Stoneking et al., Genome Res. 7:1061-1071, 1997; Zietkiewicz et al., Proc. Natl. Acad. Sci. USA 89:8448-8451, 1992).

The human genomic DNA used to make genomic subtraction samples is purified by standard methods (Ausubel et al., 1987, supra). Forensic samples are prepared for amplification by applying a protocol that is appropriate for the type of sample, as was detailed previously (Lincoln et al., “Forensic DNA Profiling Protocols,” In Methods in Molecular Biology (Humana Press, Totowa, N.J.) 1998). Alu-PCR reactions are carried out using the method of Zietkiewicz et al. (1992, supra), with the modification that PCR amplification of the DNA to be used as the “+” genomic difference sample and for the forensic samples is carried out using 5′-end biotinylated oligonucleotide primers.

Isolating ID sequences and constructing a detection ensemble array. A family of human ID sequences, defined by the enriched genomic difference sequences described above, is isolated by genomic subtraction (Straus et al., 1990, supra). Enriched genomic difference samples are prepared as is described above using samples from individuals or by pooling Alu-PCR products from several individuals (the samples may be grouped by genetic and/or regional criteria). The genomic difference sequences are cloned, sequenced, and amplified as was described previously (Rosenberg et al, 1994, supra; Straus et al., 1990, supra). To construct the detection ensemble array, the amplified subtraction products, which are genomic difference sequences, are arrayed on a nylon membrane using the robotic-based methodology of Maier et al. (J. Biotechnol. 35:191-203, 1994).

Fingerprinting a forensic sample. Forensic samples are prepared for fingerprinting by methods described previously (Lincoln, 1998, supra). A fingerprint of the human DNA in a forensic sample is obtained by hybridizing the sample's biotinlylated Alu-PCR amplification products to the detection ensemble array. The hybridization reaction (1 M NaCl/50 MM EPPS/2 mM EDTA, pH 8) is carried out for 30 minutes at 65° C. in a volume that is generally less than 1 ml. The unbound amplification products are removed by five 30 second washing steps (with shaking) at 65° C. in 2 ml wash buffer (50 mM NaCl/50 mM EPPS/2 mM EDTA, pH 8). The fingerprint (pattern of hybridization) is visualized using the Phototope-Star detection system (New England Biolabs), according to the manufacturer's recommendations.

### EXAMPLE 5

### Scanning a Sample for Numerous Human Genetic Markers

An important goal of modem medical genetics and pharmacogenomics is to obtain rapidly genomic profiles of patients. Genetic markers can be an early warning of disease (e.g., breast cancer or Huntington's disease) or can indicate to which medications a patient is likely to respond favorably. This example demonstrates the use of the genomic profiling assay for surveying the genotypes of a large number of human genetic markers in one rapid hybridization-based test.

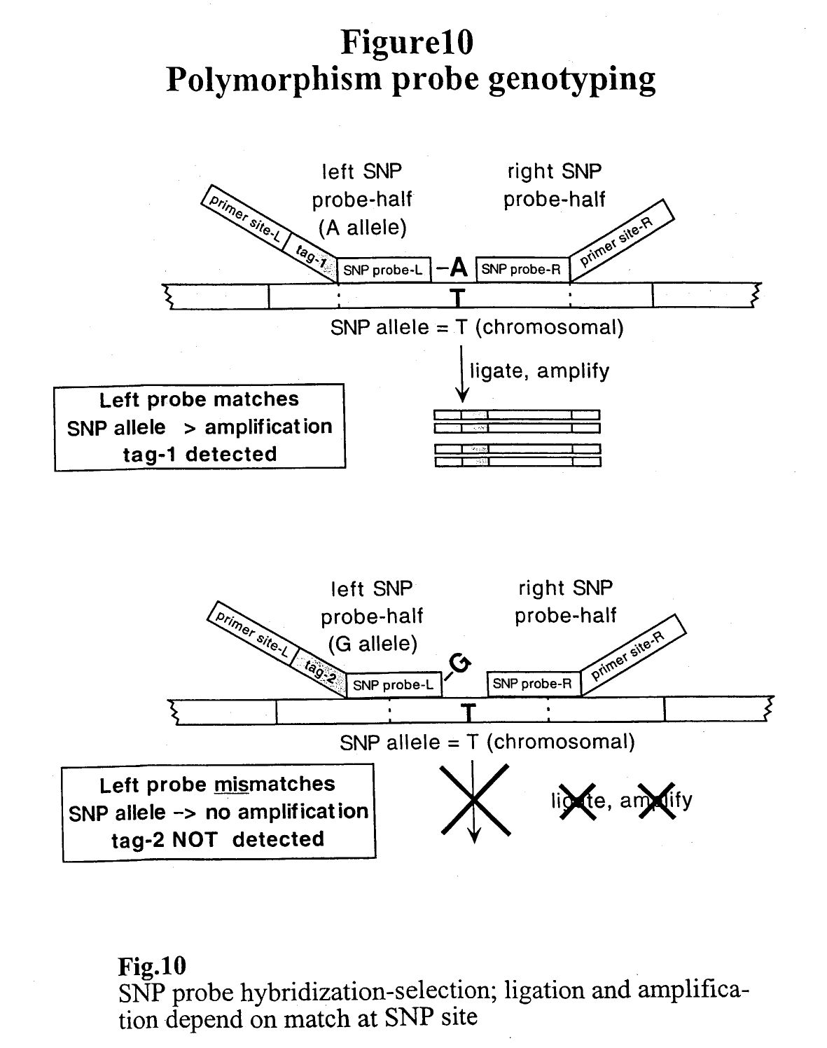

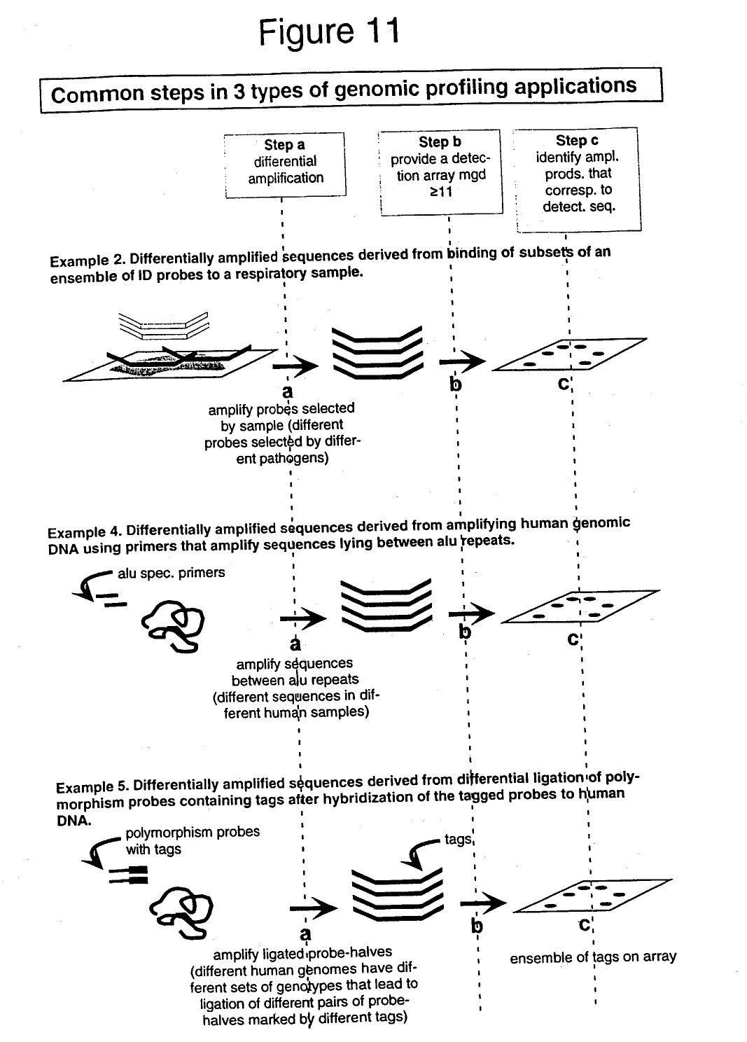

Overview of the example. In this example, a human genome is surveyed for the genotypes at numerous polymorphic sites simultaneously. As in the first three examples, an ensemble of probes, in this case SNP probes, is hybridized to genomic DNA. As before, selective amplification of the ensemble of probes generates a diagnostically informative subset of the ensemble. The members of the amplified subset are then identified by hybridization to a detection array. In this example, in contrast to previous examples, selective amplification is achieved by selective ligation of the SNP probe-halves, based on the particular SNP alleles occurring in the sample genome. The method of genotyping using SNP probes is diagrammed in FIG. 10.

Synthesizing polymorphism probe ensembles and detection ensembles. In this example, known human DNA polymorphisms are used to design polymorphism probes. The polymorphism probes can be ligated when they anneal to genomic DNA with one version of an allele, but cannot be ligated and amplified when a genome contains a different version of the allele. The use of allele-specific SNP probe ligation is illustrated in FIG. 10. The targeted DNA polymorphisms can be single-nucleotide polymorphisms (SNPs) that correspond to markers used to map the human genome (e.g., Landegren et al., Genome Res. 8:769-776, 1998) or that correspond to mutations of medical importance (e.g., the single base-pair mutation that causes the inherited disease, sickle-cell anemia). Any other type of nucleic acid sequence polymorphism (including insertions, deletions, and rearrangements) can also be incorporated in the assay.

Once the DNA polymorphisms have been chosen, polymorphism probes are synthesized basically as were the ID probes made in Example 1. The preferred design of SNP probes exploits the ability of T4 DNA ligase to discriminate against a single base-pair mismatch at the 3′ end to be ligated. In this example, however, the polymorphism probe-halves are designed so that the pairs abut at the site of the DNA polymorphism. Two polymorphism probes are generally synthesized corresponding to each targeted DNA polymorphism: one probe detects one genotype at the polymorphic site and the other probe detects the other possible genotype. Additional polymorphism probes are synthesized for loci at which several genotypes occur.

Thus, for each SNP to be genotyped, the SNP probe comprises several probe-halves. One probe-half (the right-probe half in FIG. 10) is invariant. Several versions of the left SNP probe-half are also incorporated in the assay. Each version has a different 3′ terminal nucleotide corresponding to an allele at the genomic SNP site. Only the left probe-halves that match the genomic alleles at the 3′ site will be ligated and subsequently amplified. As in the earlier examples, the amplified products can be labeled by using biotinylated primers in the amplification reaction.

Because each distinct left probe-half has a unique tag (see FIG. 10), it is possible to detect which alleles have been ligated and successfully amplified by hybridizing the labeled, amplified SNP probes to a detection array comprising an ensemble of tags that is congruent to the original ensemble of SNP probes. That is, each tag in the array corresponds to a tag (or its reverse complement) in one of the left SNP probe-halves in the original ensemble of SNP probes.

The detection array is constructed as in Example 1, except that in this case the elements of the array are the tag sequences corresponding to the polymorphism probe ensemble.

Selective amplification of human DNA polymorphisms and fingerprint analysis. Samples containing human DNA are prepared as in Example 4. If purified DNA is used, it is simply spotted on a nylon filter in 0.5 M NaOH, allowed to air dry, and crosslinked to the filter with UV light (using the Stratalinker apparatus from Stratagene according to the manufacturer's specifications). Note that as with forensic samples it may be useful to pre-amplify a sample of DNA, that is, to make a genomic representation. For example DNA from a single human hair follicle could be amplified using the Alu-PCR method described in Example 4. When a representation is used as a sample to test for SNP polymorphisms, the SNP probes are designed to correspond to polymorphisms in segments that are amplified from all samples. (Note the contrast with the previous example, in which the diagnostically useful sequences are the differentially amplified sequences which are ID probes).

The ensemble of polymorphism probes is hybridized to the sample, washed, ligated, amplified, labeled, hybridized to the detection array, and the fingerprint is visualized as is described in Example 1 (for the ID probes in that example). The pattern of hybridization to the detection array indicates the alleles represented in the genomic DNA of the sample for each polymorphic locus surveyed by the polymorphism probe ensemble.

### EXAMPLE 6

### Scanning a Cerebrospinal Fluid Sample for a Large Number of Viruses

Overview of the example. Infection of the central nervous system (CNS) is considered to be a medical emergency. Rapid diagnosis of the infectious agent is critical for optimum therapeutic outcome. Diagnosis of viral infection is particularly problematic and often expensive. The method described in this example can be used to test a cerebrospinal fluid (CSF) sample simultaneously for the presence of various types of viruses. Virus-specific ID sequences are selected in a CSF sample by solution phase hybridization-capture with an ensemble of ID probes, followed by amplification of the sample-selected ID probes. The amplified ID probes are used to probe a detection ensemble array to determine which, if any, viruses are present. The example describes a test for viruses in CSF, but a similar test can be carried out on other types of samples, including blood and solid tissue samples, following appropriate sample preparation.

Assembling ensembles of viral-specific ID sequences, probes, and primers. Group-specific sequences are chosen that are specific for each of the groups of viruses in the panel of viruses listed in Table 8. In some cases, viral-specific ID sequences are already described in the literature. In other cases, sequences are chosen from viral genomic sequences in public databases after comparing the sequences to other viruses in the database. Sequence comparisons are made using standard methods (Ausubel et al., 1987, supra). Viral-specific sequences of at least 30 bases are chosen, and corresponding ensembles of ID probes and primers are synthesized as is described in Example 3 (bloodstream pathogen assay) and as is depicted in FIGS. 9A-9C. Rather than the small mass spectrometry detection oligonucleotides depicted in FIG. 9C, however, I synthesize longer (about 20 bases) detection ensemble oligonucleotides that are complementary to the gap probes. Detection ensemble arrays are constructed by photolithography, as is described in Example 2. Positive and negative control probes are synthesized and employed as is described in Example 3.

TABLE 8
Viruses that cause CNS infections.

| | |
|---|---|
| coxsakievirus A | coxsakievirus B |
| herpes simplex virus | Togavirus |
| St. Louis encephalitis virus | measles virus |
| Epstein-Barr virus | Hepatitis |
| myxovirus | paramyxovirus |
| JC virus | mumps virus |
| Echovirus | equine encephalitis virus |
| Bunyavirus | Lymphocytic choriomeningitis virus |
| Cytomegalovirus | rabies virus |
| Varicella-zoster virus | BK virus |
| HIV | |

Scanning a sample for members of the viral panel. Preparation of CSF samples, hybridization to the ensemble of probes, purification of target sequences by magnetic separation, ligation of the selected probes, and amplification is performed as is described in Example 3. The biotinylated amplification products are then hybridized to the viral detection ensemble array and visualized as is described in Example 4.

Other Embodiments are Within the Following Claims.

What is claimed is:

1. A method for obtaining genetic information from a biological sample potentially comprising target nucleic acid molecules said method comprising the steps of:
a) providing nucleic acid molecules that are (i) target nucleic acid molecules in said sample, or (ii) probes that hybridize to target nucleic acid molecules in said sample, or (iii) amplification products of (i) or (ii), or (iv) a genomic representation of (i); and
b) detecting target nucleic acid molecules by contacting or comparing the nucleic acid molecules of (a) with a detection ensemble that has a minimum genomic derivation of greater than five and that comprises detection sequences that can detect target nucleic acid molecules.

2. The method of claim 1, further comprising the step of (c) identifying nucleic acid molecules detected in step (b).

3. The method of claim 1, wherein the detection ensemble has a minimum genomic derivation of greater than 11.

4. The method of claim 1, wherein the nucleic acid molecules of step (a) are not immobilized as size fractionated fragments in a matrix or on a solid support.

5. The method of claim 1, further comprising using fewer than four pairs of amplification sequences, to yield, if target nucleic acid molecules are present in the sample, amplification products.

6. The method of claim 5, wherein amplification is carried out using a single pair of amplification sequences.

7. The method of claim 1, wherein said method is used to quantify a target organism in said biological sample by in situ hybridization.

8. The method of claim 1, wherein prior to step (a), nucleic acid molecules of said sample are hybridized, simultaneously, with an ensemble of ID probes to yield the probes of step (a)(ii).

9. The method of claim 1, wherein the probes of step (a)(ii) include (i) a first region capable of hybridizing to a target nucleic acid molecule, and (ii) amplification sequences.

10. The method of claim 1, wherein said nucleic acid molecules of said sample are fixed to a solid support.

11. The method of claim 1, wherein said nucleic acid molecules of step (a) are in the liquid phase.

12. The method of claim 1, wherein at least some of the nucleic acid molecules of step (a) comprise one or more oligonucleotide tags.

13. The method of claim 1, wherein at least some of the probes of step (a)(ii) comprise: (i) two or more oligonucleotides that can be ligated to one another upon hybridization to a target nucleic acid molecule, and (ii) amplification sequences.

14. The method of claim 1, wherein said detection sequences of said detection ensemble are arrayed as spots in two dimensions or as parallel stripes on a solid support.

15. The method of claim 8, wherein said ensemble of ID probes includes probes that hybridize to at least two different nucleic acid molecules from each of at least ten different viruses, each of which belongs to a different genus.

16. The method of claim 1, wherein said biological sample is a gastrointestinal tract sample, and said genetic information is the identification of nucleic acid molecules in said sample from 6 or more of Escherichia coli, Salmonella, Shigella, Yersinia enterocolitica, Vibrio cholera, Campylobacter fecalis, Clostridium difficile, Rotavirus, Norwalk virus, Astrovirus, Adenovirus, Coronavirus, Giardia lamblia, Entamoeba histolytica, Blastocystis hominis, Cryptosporidium, Microsporidium, Necator americanus, Ascaris lumbricoides, Trichuris trichiura, Enterobius vermicularis, Strongyloides stercoralis, Opsthorchis viverrini, Clonorchis sinensis, and Hymenoplepis nana.

17. The method of claim 1, wherein said biological sample is a respiratory tract sample, and said genetic information is the identification of nucleic acid molecules in said sample from 6 or more of Cornybacterium diphtheriae, Mycobacterium tuberculosis, Mycoplasma pneumoniae, Chlamydia trachomatis, Chlamydia pneumoniae, Bordetella pertussis, Legionella spp., Nocardia spp., Streptococcus pneumoniae, Haemophilus influenzae, Chlamydia psittaci, Pseudomonas aeruginosa, Staphylococcus aureus, Histoplasma capsulatum, Coccidoides immitis, Cryptococcus neoformans, Blastomyces dermatitidis, Pneumocystis carinii, Respiratory Syncytial Virus, Adenovirus, Herpes simplex virus, Influenza virus, Parainfluenza virus, and Rhinovirus.

18. The method of claim 1, wherein said biological sample is a blood sample, and said genetic information is the identification of nucleic acid molecules in said sample from 6 or more of Coagulase-negative staphylococci, Staphylococcus aureus, Viridans streptococci, Enterococcus spp., Beta-hemolytic streptococci, Streptococcus pneumoniae, Escherichia spp., Klebsiella spp., Pseudomonas spp., Enterbater spp., Proteus spp., Bacteroides spp., Clostridium spp., Pseudomonas aueruginosa, Cornybacterium spp., Plasmodium spp., Leishmania donovani, Toxoplasma spp., Microfilariae, Fungi, Histoplasma capsulatum, Coccidoides immitis, Cryptococcus neoformans, Candida spp., HIV, Herpes simplex virus, Hepatitis C virus, Hepatitis B virus, Cytomegalovirus, and Epstein-Barr virus.

19. The method of claim 1, wherein said genetic information is the identification of nucleic acid molecules in said sample from 6 or more of coxsakievirus A, Herpes simplex virus, St. Louis encephalitis virus, Epstein-Barr virus, myxovirus, JC virus, coxsakievirus B, togavirus, measles virus, a hepatitis virus, paramyxovirus, echovirus, bunyavirus, cytomegalovirus, varicella-zoster virus, HIV, mumps virus, equine encephalitis virus, lymphocytic choriomeningitis virus, rabies virus, and BK virus.

20. The method of claim 8, wherein at least 50% of the probes comprising said ensemble of nucleic acid probes are capable of hybridizing to pre-determined genomic difference sequences that are potentially present in said sample or in a genomic representation of said sample.

21. A kit for obtaining genetic information from a biological sample, comprising:
a) a plurality of ID probes and/or SNP probes; and
b) a detection ensemble comprising detection sequences that are congruent with probes of (a), wherein said detection ensemble has a minimum genomic derivation of greater than five.

22. The kit of claim 21, wherein (a) comprises more than ten different amplifiable probes.

23. The kit of claim 22, wherein (a) comprises more than fifty different amplifiable probes.

24. The kit of claim 23, wherein (a) comprises more than two hundred and fifty different amplifiable probes.

25. The kit of claim 21, wherein the detection ensemble has a minimum genomic derivation of greater than 11.

26. The kit of claim 21, wherein (a) comprises more than five families of amplifiable probes.

27. The kit of claim 21, wherein the probes of (a) are specific for at least two distinct taxa.

28. The kit of claim 27, wherein the probes of (a) are specific for at least two different species.

29. The kit of claim 27, wherein the probes of (a) are specific for at least two different genera.

30. The kit of claim 27, wherein the probes of (a) are specific for at least two different kingdoms.

31. The kit of claim 21, wherein the probes of (a) include probes that comprise: (i) two or more oligonucleotides that can be ligated to one another upon hybridization to an ID sequence of a target nucleic acid molecules, and (ii) amplification sequences.

32. The kit of claim 21, wherein the probes of (a) and/or the detection sequences of (b) are physically attached to distinct locations on a solid support.

33. The kit of claim 21, wherein at least 50% of the probes of (a) comprise genomic difference sequences from at least three different species.

34. The kit of claim 32, in which the detection sequences comprised by the detection ensemble that detect (i) members of a taxonomic group and (ii) closely related taxonomic groups are positioned adjacent to one another on said support.

35. An ensemble of ID probes that can be amplified using fewer than four pairs of amplification sequences and that comprises more than three families of ID probes and more than ten different ID probes.

36. The ensemble of claim 35, comprising more than fifty different amplifiable ID probes.

37. The ensemble of claim 36, comprising more than two hundred and fifty different amplifiable ID probes.

38. The ensemble of claim 35, comprising more than ten families of amplifiable ID probes.

39. The ensemble of claim 35, comprising more than twenty-five families of amplifiable ID probes.

40. The ensemble of claim 35, wherein more than two of said families of amplifiable probes are specific for non-overlapping taxa.

41. The ensemble of claim 35, wherein more than two of said families of amplifiable probes are specific for different species.

42. The ensemble of claim 35, wherein more than two of said families of amplifiable probes are specific for different genera.

43. The ensemble of claim 35, wherein more than two of said families of amplifiable probes are specific for different kingdoms.

44. The ensemble of claim 35, wherein the probes of (a) include probes that comprise: (i) two or more oligonucleotides that can be ligated to one another upon hybridization to an ID sequence of a target nucleic acid molecule, and (ii) amplification sequences.

45. The ensemble of claim 35, wherein at least 50% of said probes comprise genomic difference sequences from at least three different species.

46. The ensemble of claim 35, in which the detection sequences comprised by the detection ensemble that detect (i) members of a taxonomic group and (ii) closely related taxonomic groups are positioned adjacent to one another on a support.

47. A method for obtaining genetic information from a biological sample potentially comprising target nucleic acid molecules, said method comprising the steps of:
a) providing an ensemble of nucleic acid probes having a minimum genomic derivation of greater than five;
b) contacting said ensemble of probes, simultaneously, with nucleic acid molecules of said sample;
c) detecting hybridization between said probes and any target nucleic acid molecules of said sample; and
d) identifying nucleic acid molecules detected in step (c).

48. The method of claim 13, wherein said oligonucleotides that can be ligated are SNP probes.

49. The method of claim 48, wherein at least some of said SNP probes comprise tag sequences that can hybridize to tag sequences in a detection ensemble comprising an ensemble of tag sequences congruent to said SNP probes.

50. The method of claim 48, wherein the detection ensemble has a minimum genomic derivation of greater than 20.

51. The method claim 50, wherein the detection ensemble has a minimum genomic variation of greater than 50.

52. The method of claim 1, wherein the amplification products of step (a)(iv) are generated by amplification of target nucleic acid molecules of step (a)(i) using no more than four pairs of amplification sequences.

53. The method of claim 52, wherein said amplification sequences direct the amplification of sequences lying between Alu repeats using Alu-specific primers.

54. The method of claim 52, wherein the detection ensemble of (b) comprises ID sites that are congruent to ID probes potentially amplified in step (a)(iv).

55. A kit for obtaining genetic information from a biological sample, comprising
a) a plurality of nucleic acid primers that are capable of priming the amplification of DNA sequences flanked by repetitive sequences in target genomic DNA in a biological sample to yield ID probes; and
b) a detection ensemble comprising detection sequences that are congruent with ID probes potentially amplified using the primers of (a), wherein said detection ensemble has a minimum genomic derivation of greater than five.

56. The kit of claim 55, wherein said detection ensemble has a minimum genomic derivation of greater than 20.

57. The kit of claim 55, wherein said repetitive sequences are human Alu repeats, and said primers are Alu-specific primers.


SEQUENCE LISTING (5 sequences)

SEQ ID NO: 1 - DNA, 24 bp, Artificial Sequence
  primer
gacactctcgagacatcaccgtcc

SEQ ID NO: 2 - DNA, 22 bp, Artificial Sequence
  primer
gttggtttaaggcgcaagaatt

SEQ ID NO: 3 - DNA, 39 bp, Artificial Sequence
  primer
gacactctcgagacatcaccgtccnnnnnnnnnnnnnnn

SEQ ID NO: 4 - DNA, 37 bp, Artificial Sequence
  primer
nnnnnnnnnnnnnnngttggtttaaggcgcaagaatt

SEQ ID NO: 5 - DNA, 22 bp, Artificial Sequence
  primer
aattcttgcgccttaaaccaac